(19) United States
(12) Patent Application Publication
(10) Pub. No.: US 2021/0292390 A1
(43) Pub. Date: Sep. 23, 2021

(54) CHIMERIC ANTIGEN RECEPTORS

(71) Applicant: Oxford University Innovation Limited, Oxford (GB)

(72) Inventors: Johannes BREUNING, Oxford (GB); Marion H. BROWN, Oxford (GB)

(21) Appl. No.: 16/321,651

(22) PCT Filed: Aug. 4, 2017
(86) PCT No.: PCT/GB2017/052314

§ 371 Date: Jan. 29, 2019

(30) Foreign Application Priority Data
Aug. 4, 2016 (GB) 1613473.6

(51) Int. Cl. C07K 14/705; A61P 35/00; A61K 35/17; C07K 14/725; C07K 16/28

(52) U.S. Cl. CPC C07K 14/70596; A61P 35/00; A61K 35/17; C07K 14/7051; C07K 16/2803; C07K 14/70517; A61K 38/00; C07K 14/70521; C07K 2319/02; C07K 2319/03; C07K 2319/30; C07K 2319/33; C07K 14/70578

(57) ABSTRACT

Disclosed are chimeric antigen receptors, and cells modified to express one or more proteins comprising regions of a chimeric antigen receptor (CAR). The CARs comprise an antigen binding region, a transmembrane region and a CD6-derived signalling region, wherein the CD6-derived signalling region comprises a GADS binding motif and a SLP-76 binding motif. The CD6-derived signalling region may be between 20 and 60 amino acid residues in length. The CARs exhibit increased cell activation and increased target cell killing when incorporated in CAR T cells. Also disclosed are nucleic acids encoding such CARs, and collections of nucleic acids. There are also provided medical uses of the CARs, cells, or nucleic acids, and methods of treating a condition or infection.

### FIELD OF THE INVENTION

The present invention relates to chimeric antigen receptors. The invention also relates to cells modified to express one or more proteins comprising regions of a chimeric antigen receptor. The invention further relates to nucleic acids encoding such chimeric antigen receptors, and to collections of nucleic acids. The present invention relates to methods of treating a condition or infection.

### INTRODUCTION

Adoptive immunotherapy for the treatment of cancer or infections has undergone a significant resurgence in clinical activity. One of the most promising immunotherapy approaches for the treatment of tumours is the use of a T cell expressing a chimeric antigen receptor (CAR) (Cheadle, 2014). CARs are recombinant receptors that target surface molecules, and are typically composed of an extracellular antigen binding region that is linked via a linker/hinge and transmembrane regions to an intracellular signalling region (Maus et al., 2013).

CARs typically contain an extracellular single chain variable fragment (scFv) from an antibody that binds to antigens on the surface of tumour cells and a cytoplasmic region providing activating signals to the T cell. The design of the cytoplasmic region of CARs is critical for CAR T cell function, producing downstream signalling that is strong enough for T cell activation whilst avoiding triggering a cytokine storm.

CARs were first developed in the 1980s, since then several generations of CARs have been produced. The first generation of CAR T cells contain a scFv against a cell surface antigen expressed on a tumour cell and an intracellular CD3 zeta chain signalling region. These first generation CARs failed to produce sustained anti-tumour effects.

The second generation of CARs contain a scFv and constructs with signalling regions from CD3 zeta chain and an additional signalling region from CD28, ICOS, OX-40 or 4-1BB in succession with the CD3 zeta chain region.

Third generation CARs contain the CD3 zeta chain, a signalling region from CD28 and an OX-40 or 4-1BB signalling region, providing a full complement of activation, proliferation and survival signals. It has not been proven that third generation CARs are more efficient than second generation CARs (Milone et al., 2009). Indeed, there are some disadvantages of third generation CARs, for example, “off-target” binding can occur triggering potent activation signals and a potentially lethal cytokine storm. Furthermore, reduction of the signal threshold results in activation in the absence of the triggering antigen (Brudno and Kochenderfer, 2016).

A fourth generation of CARs have been described, CAR T cells redirected for cytokine killing (TRUCKS) where the vector containing the CAR construct possesses a cytokine cassette. When the CAR is ligated, the CAR T cell deposits a pro-inflammatory cytokine into the tumour lesion (Cheadle, 2014).

Unlike naïve T cell receptors, CARs recognised unprocessed antigens regardless of their expression of major histocompatibility antigens. CAR T cells can circumvent some of the mechanisms by which tumours avoid MHC-restricted T cell recognition (Maus et al., 2013).

CD6 is a 105-130 kDa transmembrane glycoprotein expressed at the surface of primary T cells. CD6 regulates T cell activation in a ligand dependent manner through binding of another cell surface receptor, CD166 for full T cell activation (Hassan et al 2004, Hassan et al. 2006 Chappell et al. 2015). CD6 has a long cytoplasmic tail, (244 amino acids) that interacts with the adaptor protein SLP-76 which is important for costimulation (Hassan, 2006). The function of CD6 has been attributed both stimulatory and inhibitory functions (Hassan et al., 2004, Hassan et al., 2006), and it has been reported that CD6 can inhibit early TCR signalling (Oliveira et al., 2012).

### SUMMARY OF THE INVENTION

According to a first aspect of the present invention, there is provided a chimeric antigen receptor (CAR) comprising an antigen binding region, a transmembrane region and a CD6-derived signalling region, wherein the CD6-derived signalling region comprises a GADS binding motif and a SLP-76 binding motif.

According to a second aspect of the present invention, there is provided a cell modified to express one or more proteins comprising regions of a chimeric antigen receptor (CAR), said regions jointly comprising:
an antigen binding region;
a transmembrane region;
a CD6-derived signalling region comprising a GADS binding motif; and
a CD6-derived signalling region comprising a SLP-76 binding motif.

According to a third aspect of the present invention, there is provided a nucleic acid sequence encoding the CAR of the first aspect. Cellular expression of nucleic acids of the third aspect of the invention represents a suitable method for the production of CARs of the first aspect of the invention.

According to a fourth aspect of the present invention, there is provided a collection of nucleic acid sequences encoding the one or more proteins of a cell of the second aspect of the invention. The collection of nucleic acid sequences may be provided in the form of a collection of nucleic acid molecules. The collection of nucleic acid sequences may be provided in the form of one or more expression vectors comprising the nucleic acid sequences. Cellular expression of a collection of nucleic acids in accordance with the fourth aspect of the invention represents a suitable method for the production of cells in accordance with the second aspect of the invention.

According to a fifth aspect of the invention, there is provided a method of treating a condition in a subject in need thereof, the method comprising providing the subject with a CAR in accordance with the first aspect of the invention. The CAR may be provided in a cell modified to express the CAR of the first aspect of the invention. The CAR may be provided by cellular expression of a nucleic acid sequence of the third aspect of the invention.

According to a sixth aspect of the invention, there is provided a method of treating a condition in a subject in need thereof, the method comprising providing the subject with a cell in accordance with the second aspect of the invention. The cell in accordance with the third aspect of the invention may be provided by cellular expression of a collection of nucleic acids in accordance with the fourth aspect of the invention.

The methods of treatment of the fifth and sixth aspects of the invention are of particular utility in the treatment of cancer, though they may also be used in treatment of other conditions, as described further below.

### DESCRIPTION OF THE FIGURES

The invention is further illustrated by the accompanying Figures, in which:

FIG. 1. Illustrates the role of CD6 and Y662 in Jurkat activation after CD3 and CD6 antibody stimulation, measured by Mean Florescence Intensity (MFI) of CD69, a T cell activation marker.

FIG. 2. Shows co-immunoprecipitation of CD6 mutants, demonstrating a cooperation of CD6 Y629 and Y662 and the quantification of Western blot bands of three independent co-immunoprecipitations. The order of the bars on the graphs in panel B read left to right is the same as the order of the key read top to bottom.

FIG. 3. Shows graphs demonstrating the activating effect of CD6 in primary T cells assessed by CD69 expression is mediated by the Y629 and Y662 residues.

FIG. 4. Illustrates the activating effect of CD6 on IL-2 production, a marker of T cell activation, in primary T cells is mediated by the CD6 Y629 and Y662. The order of the bars on the graphs read left to right is the same as the order of the key read top to bottom.

FIG. 5. Illustrates the effects on T cell activation of incorporating a CD6-derived signalling region comprising a GADS binding motif and SLP-76 binding motif from CD6 into a chimeric receptor. Panel A shows a graph illustrating expression of the comparator “Zeta” and exemplary “Zeta+CD6 C” chimeric receptors in 2B4 Reay hybridoma T cells. Isotype control is the peak to the left, while the peaks for Zeta and Zeta+CD6C on the right are largely superimposed on one another. Panel B shows the effects of CD6 incorporation on IL-2 concentration in response to CD6 crosslinking demonstrated as a log scale. Panel C shows the effects of CD6 incorporation on IL-2 concentration in response to CD6 crosslinking demonstrated as linear scale.

FIG. 6. Shows 4-1BB zeta and 4-1BB zeta CD6 CAR expression on CD4+ and CD8+ T cells. CAR T cells were stained with a Fab-specific anti-mouse IgG biotin antibody and streptavidin APC. Shown is the staining of T cells from one example donor. Both CARs of the invention, and control CARs from which these were derived, are expressed at similar levels.

FIG. 7. Shows graphs illustrating cytokine production by CD4+ CAR T cells in response to incubation with Daudi cells. Adding the CD6 C terminus to a 4-1BB zeta CAR increases the production of IFNγ but not IL-2. 10⁵ donor 1 CD4+ CAR T cells were incubated with the indicated amounts of Daudi cells overnight and IL-2 (A) and IFNγ (B) secretion was measured by ELISA. Incubation of CD4+ CAR T cells with CD19− Jurkat cells did not result in cytokine production (data not shown). Results from two independent experiments with duplicates in each experiment, *p<0.05, **p<0.01 Student's t test for the indicated amounts of Daudi cells.

FIG. 8. Shows graphs illustrating cytotoxic granule exocytosis by CD8+ CAR T cells in response to Daudi cells. Adding the CD6 C terminus to a 4-1BB zeta CAR enhances cytotoxic granule exocytosis. 5×10⁴ donor 1 and 2 CD8+ CAR T cells were incubated for 5 hours with varying amounts of Daudi cells in the presence of a CD107a antibody and Monensin and the mean fluorescence intensity (MFI) was measured using flow cytometry. Incubation of CD8+ CAR T cells with CD19− Jurkat cells did not result in any CD107a response (data not shown). Results from two independent experiments with duplicates in each experiment, *p<0.05, **p<0.01, Student's t test.

FIG. 9. Shows graphs illustrating target cell lysis (Daudi cells) in response to incubation with CD8+ CAR T cells. Adding the CD6 C terminus to a 4-1BB zeta CAR enhances tumour cell killing. Daudi and Jurkat cells were labelled with the cell dye CFSE and incubated for 6 hours with donor 1 and 2 CD8+ CAR T cells. Specific lysis was measured by assessing the ratio of Daudi cells to Jurkat cells. Results from two independent experiments with duplicates in each experiment, *p<0.05, **p<0.01, Student's t test for the indicated ratios of T cells to target cells.

FIG. 10. Shows target cell lysis measured by LDH release in response to CD8+ CAR T cells. T cells were incubated with Daudi cells at varying ratios for 6 hours. The release of LDH is the measured using the CytoTox 96 non-radioactive cytotoxicity assay (Promega). Incubation of CD8+ CAR T cells with CD19− Jurkat cells did not result in LDH release (data not shown). Results from one experiment with triplicates in each experiment.

Table 1. Sets out results demonstrating that the GADS SH2 domain specifically binds to CD6 pY629 and that the SLP-76 SH2 domain specifically binds to CD6 pY662.

### DETAILED DESCRIPTION OF THE INVENTION

The present invention is based upon the inventors' finding that when the GADS binding motif and SLP-76 binding motif from CD6 are incorporated into CARs, the activation that can be achieved in respect of T-cells comprising such CARs is markedly increased. As demonstrated in the Examples, cells comprising the CARs of the invention exhibit elevated expression of certain cytokines, such as interferon-gamma, and also significantly enhanced killing of target cells.

The inventors' opinion is that preservation of the spatial arrangement of the GADS binding motif and SLP-76 binding motif that is found in native CD6 protein may be helpful in bringing about the increase in activation of T-cells, and in achieving maximal levels of activation.

Prior to the present disclosure, the binding of GADS to CD6 had not been known. The binding motif identified by the inventors in CD6 is YQNF, which corresponds to YXN motif found in the binding of SH2 domains. Such motifs are not exclusive to CD6 but are found in a wide range of molecules. The identification of the importance of the GADS binding site Y629 in T cell activation, and the cooperation of Y629 and Y662 in recruiting GADS and SLP-76 associated with T cell activation, is a new finding that gives rise to the novel and inventive applications (in CARs, cells, nucleic acids, and methods of treatment) described herein.

The increase in activation observed is only achieved in respect of CARs in which the GADS binding motif and SLP-76 binding motif are provided in a CD6-derived signalling region that is truncated as compared with the full length cytoplasmic region of CD6.

Fusion proteins comprising CARs and the full length (244 amino acid) cytoplasmic region of CD6 are subject to compromised cellular expression. Without wishing to be bound by any hypothesis, the inventors believe that it is likely the large size of the complete cytoplasmic region of CD6 that causes this effect. Furthermore, parts of the cytoplasmic tail of CD6 may have inhibitory functions which will reduce the activity of CARs in which they are utilised.

In contrast, the inventors have found that addition to a first generation CAR of a truncated sequence comprising the GADS binding motif and SLP-76 binding motif from the signalling region of CD6 did not compromise cellular expression of the CAR, and afforded marked advantages in terms of the increased level of T-cell activation that could be attained. This was evident in both the production of certain cytokines, such as interferon-gamma, and the cells' ability to kill their targets. These advantages may be provided with the CD6-derived signalling region incorporated in first, second, third or fourth generation CARs.

### Chimeric Antigen Receptors

As set out above, chimeric antigen receptors (CARs) are engineered transmembrane chimeric proteins designed to assign antigen specificity to T-cells. They are recombinant receptors comprising an antigen binding region, a transmembrane region and an intracellular signalling region. These regions are described in more detail later in the specification.

To demonstrate the utility of the CARs and cells of the invention, the inventors first produced a chimeric receptor that includes all intracellular constituents of such a CAR (the only region lacking being an extracellular antigen binding region). As discussed above, the cytoplasmic regions of CARs are essential for their activity in therapeutic contexts.

The sequence of this exemplary chimeric receptor is set out in SEQ ID NO. 4. This experimental chimeric receptor comprises both human and murine constituents. As illustrated further in the Examples, this experimental chimeric receptor is illustrative of CARs in accordance with the present invention, as exemplified by the experimental chimeric receptor of SEQ ID NO. 4, which are able to markedly increase T-cell activation when expressed by appropriate cells. Merely by way of example, the chimeric receptor of SEQ ID NO.4 is able to increase IL-2 production at least 2.5 fold as compared to a control receptor lacking the CD6-derived signalling region.

The inventors also produced a CAR of the invention comprising not only the intracellular constituents of a CAR, but also an extracellular antigen binding region comprising an scFv fragment from an antibody that binds to CD19 antigens on the surface of tumour cells. DNA encoding this CAR of the invention is shown in SEQ ID NO: 11, and the amino acid sequence of this CAR of the invention is set out in SEQ ID NO: 13.

A CAR of the invention may share at least 80%, at least 85%, at least 90%, at least 91%, at least 92%, at least 93%, at least 94%, at least 95%, at least 96%, at least 97%, at least 98%, or at least 99% identity with the amino acid sequence of the exemplary CAR set out in SEQ ID NO: 13. A CAR of the invention may comprise the amino acid sequence of the exemplary CAR set out in SEQ ID NO: 13. A CAR of the invention may consist of the amino acid sequence of the exemplary CAR set out in SEQ ID NO: 13.

A CAR of the invention may share at least 75%, at least 80%, at least 85%, at least 90%, at least 91%, at least 92%, at least 93%, at least 94%, at least 95%, at least 96%, at least 97%, at least 98%, or at least 99% identity with the non-antigen-binding portion of the amino acid sequence of the exemplary CAR set out in SEQ ID NO: 13. A CAR of the invention may comprise the amino acid sequence of the non-antigen-binding portion of the exemplary CAR set out in SEQ ID NO: 13. A CAR of the invention may consist of the amino acid sequence of the non-antigen-binding portion of the exemplary CAR set out in SEQ ID NO: 13.

A CAR of the invention may share at least 75%, at least 80%, at least 85%, at least 90%, at least 91%, at least 92%, at least 93%, at least 94%, at least 95%, at least 96%, at least 97%, at least 98%, or at least 99% identity with the cytoplasmic portion of the amino acid sequence of the exemplary CAR set out in SEQ ID NO: 13. A CAR of the invention may comprise the amino acid sequence of the cytoplasmic portion of the exemplary CAR set out in SEQ ID NO: 13. A CAR of the invention may consist of the amino acid sequence of the cytoplasmic portion of the exemplary CAR set out in SEQ ID NO: 13.

### CD6

The CD6 upon which the CD6-derived signalling regions are based may be any mammalian CD6. However, human CD6 is a preferred protein from which such regions may be derived.

The sequence of a wild type human CD6 protein is set out in SEQ ID NO. 1. The cytoplasmic signalling region of the wild type human CD6 protein comprises amino acid resides 425 to 668 of SEQ ID NO. 1, and is set out in SEQ ID NO. 2.

### GADS and the GADS Binding Motif

GADS (GRB2-related adaptor downstream of Shc) is an adaptor protein that has an N-terminal and C-terminal Src homology 3 (SH3) domain flanking a central SH2 domain and a proline-rich region. GADS couples the T-cell receptor to distal signalling events by linking the adapter protein SLP-76 to LAT (linker for activation of T cells) by binding a proline-rich region in SLP-76 with its SH3 domain and the GADS binding motif of CD6 with its SH2 domain. Distal signalling events are believed to follow this mechanism whether SLP-76 is recruited to LAT or to CD6.

The inventors have identified the GADS binding motif present in CD6 as comprising the tyrosine (Y) residue corresponding to that located at 629 of SEQ ID NO. 1. Thus, in a suitable embodiment, the GADS binding motif present in a CD6-derived signalling region in accordance with the invention, comprises at least a tyrosine residue corresponding to Y629 of SEQ ID NO. 1.

Suitably, the GADS binding motif incorporated in a CD6-derived signalling region may comprise the amino acid residues YXN corresponding to those found at positions 629-631 of SEQ ID NO. 1.

Suitably, the GADS binding motif incorporated in a CD6-derived signalling region may comprise the amino acid residues YQNF corresponding to those found at positions 629-632 of SEQ ID NO. 1. The GADS binding motif incorporated in a CD6-derived signalling region may comprise the amino acid residues YQNF at positions 629-632 of SEQ ID NO. 1. The GADS binding motif may optionally comprise up to a further one, two, three, four, five, six, seven or eight amino acid residues upstream of position 632 of SEQ ID NO: 1. Suitably the GADS binding motif may optionally comprise up to a further one, two, three, four, five, six, seven or eight amino acid residues downstream of position 629 of SEQ ID NO: 1.

### SLP-76 and the SLP-76 Binding Motif

SLP-76 (SH2 region-containing leukocyte protein of 76 kDa) is an adaptor protein that mediates signalling from the T-cell receptor.

Three tyrosine residues close to the N-terminus can be phosphorylated and recruit signalling molecules. signalling molecules such as GADS can also be recruited to the proline-rich region of SLP-76. SLP-76 can be recruited to CD6 by binding to the phosphorylated tyrosine 662 residue of CD6 with its SH2 domain.

The SLP-76 binding motif of CD6 comprises at least a tyrosine residue corresponding to Y662 of the wild type CD6 protein set out in SEQ ID NO. 1.

Suitably the SLP-76 binding motif incorporated in a CD6-derived signalling region may comprise the amino acid residues YXD at positions 662-664 of SEQ ID NO. 1

Suitably the SLP-76 binding motif incorporated in a CD6-derived signalling region may comprise the amino acid residues YDDI at positions 662-665 of the CD6 protein set out in SEQ ID NO. 1. The SLP-76 binding motif may comprise the amino acid residues YDDI at positions 662-665 of SEQ ID NO. 1. The SLP-76 binding motif may optionally comprise up to a further one, two, three, four, five, six, seven or eight amino acid residues upstream of position 665 of SEQ ID NO:1. Suitably the SLP-76 binding motif may optionally comprise up to a further one, two, three, four, five, six, seven or eight amino acid residues downstream of position 662 of SEQ ID NO:1.

### CD6-Derived Signalling Region

The CARs or cells of the invention make use of CD6-derived signalling regions incorporating GADS binding motifs and/or SLP-76 binding motifs from CD6.

In the context of the present invention, the term “CD6-derived signalling region” is intended to mean a region that is based upon the cytoplasmic signalling region of the wild type CD6 protein (for example, a fragment or variant of the cytoplasmic signalling region, as considered further below), but which does not comprise the whole of the cytoplasmic region of the wild type CD6 protein. As referred to above, CARs incorporating the whole cytoplasmic region of wild type CD6 protein do not offer the advantages observed in respect of the CARs of the invention. Suitably the CD6-derived signalling region may be the only cytoplasmic part of CD6 present in a CAR or cell of the cell of the invention.

In view of the above, it will be appreciated that the whole cytoplasmic region of CD6 does not constitute a “CD6-derived signalling region” as set out herein, and a CAR comprising the full length of the native CD6 signalling region, without any other CD6-derived portions, will not constitute a CAR in accordance with the present invention.

Except for where the context requires otherwise, the various embodiments of CD6-derived signalling regions described herein should be taken as suitable to be incorporated in the CARs of the first aspect of the invention, or to be incorporated in the cells of the second aspect of the invention.

The CD6-derived signalling region may comprise one or more fragments of the cytoplasmic region of the wild type CD6 protein. For the purposes of the present disclosure, a fragment of the cytoplasmic region of CD6 should be taken as being a truncated portion of the relevant wild type CD6 sequence, in which each of the amino acid residues present corresponds directly to the residues found in the native protein. For example, a suitable CD6-derived signalling region to be incorporated in the CARs of the first aspect of the invention, or the cells of the second aspect of the invention may comprise one or more truncated portions of SEQ ID NO. 2.

Additionally, or alternatively, the CD6-derived signalling region may comprise a variant of the cytoplasmic region of the wild type CD6 protein or of a fragment thereof. For the purposes of the present disclosure, a variant of the native CD6 cytoplasmic region, or a fragment thereof, is an amino acid sequence that shares less than 100% identity with the residues found in the native protein or fragment. As described further below, a suitable variant may share at least 70% sequence identity with the corresponding native sequence.

A suitable CD6-derived signalling region may comprise one or more fragments from the cytoplasmic region of CD6, optionally in combination with one or more variants of the native sequence, as described herein.

A CD6-derived signalling region, whether in a CAR or cells of the invention, may comprise more than one fragment or variant of wild type CD6 protein. For example, a CD6-derived signalling region may comprise two or more fragments or variants of the wild type CD6 protein, three or more fragments or variants of the wild type CD6 protein, four or more fragments or variants of the wild type CD6 protein, or five or more fragments or variants of the wild type CD6 protein.

Fragments or variants of CD6 incorporated in the CD6-derived signalling region may be joined by linker sequences that comprise one or more amino acid residues that do not form part of the contiguous sequence in the native CD6 protein.

Except for where the context requires otherwise, the following considerations set out in this disclosure with respect to fragments of CD6 suitable for incorporation in the CARs or cells of the invention should also be taken as applicable to variants based upon such fragments.

In a suitable embodiment the CD6-derived signalling region may comprise a fragment or variant of the region of the wild type CD6 protein set out in SEQ ID NO. 2.

The CD6-derived signalling region may comprise the amino acid sequence set out in SEQ ID NO. 3. This 46 amino acid residue fragment of the cytoplasmic region of CD6 is incorporated in the exemplary chimeric receptor of the invention set out in SEQ ID NO. 4, and described further in the Examples.

Any given fragment of the CD6-derived signalling region may comprise between 2 and 100 amino acids in length, between 8 and 80 amino acids in length, between 10 and 70 amino acids in length, between 20 and 60 amino acids in length, between 40 and 50 amino acids in length, between 43 and 46 amino acids in length.

Without detracting from the above, a suitable fragment of the CD6-derived signalling region may be up to 5 amino acids in length, up to 10 amino acids in length, up to 20 amino acids in length, up to 30 amino acids in length, up to 40 amino acids in length, or up to 50 amino acids in length.

Suitably a fragment of the CD6-derived signalling region may be at least 5 amino acids in length, at least 10 amino acids in length, at least 20 amino acids in length, at least 30 amino acids in length, at least 40 amino acids in length, or at least 50 amino acids in length.

Suitably a fragment for incorporation in a CD6-derived signalling region comprises a GADS binding motif. Suitably a fragment for incorporation in a CD6-derived signalling region comprises a SLP-76 binding motif. Suitably a fragment for incorporation in a CD6-derived signalling region comprises both a GADS binding motif and a SLP-76 binding motif.

The GADS binding motif and the SLP-76 binding motif may be comprised within the same fragment or different fragments of the wild type CD6 protein.

In one embodiment, the GADS binding motif is comprised within a first fragment and the SLP-76 binding motif is comprised within a second fragment. Accordingly, the CD6-derived signalling region may comprise a first and a second fragment, jointly comprising the GADS binding motif and a SLP-76 binding motif.

In another embodiment, both the GADS binding motif and the SLP-76 binding motif are comprised in a single fragment. Accordingly, the CD6-derived signalling region may comprise a single fragment comprising the GADS binding motif and the SLP-76 binding motif.

In a further embodiment, parts of the GADS binding motif and parts of the SLP-76 binding motif are comprised within multiple fragments. Accordingly, the CD6-derived signalling region may comprise multiple fragments, jointly comprising the GADS binding motif and a SLP-76 binding motif.

The CD6-derived signalling region and the transmembrane region may be separated such that the CD6-derived signalling region begins between 170 and 230, between 180 and 220, between 185 and 215, or between 190 and 210 amino acid residues from the inner surface of the cell membrane. For example, the CD6-derived signalling region may be approximately 194 amino acid residues from the inner surface of the cell membrane.

In a suitable embodiment the GADS binding motif may be positioned within a CD6-derived signalling region such that its tyrosine residue (corresponding to residue 629 of SEQ ID NO:1) is located between 180 and 230, between 190 and 220, between 195 and 215, or between 200 and 210 amino acid residues from the inner surface of the cell membrane. For example, the tyrosine residue of the GADS binding motif may be located approximately 205 amino acid residues from the inner surface of the cell membrane.

In a suitable embodiment the SLP-76 binding motif may be positioned within a CD6-derived signalling region such that its tyrosine residue (corresponding to residue 662 of SEQ ID NO:1) is located between 205 and 265, between 215 and 255, between 225 and 245, or between 235 and 240 amino acid residues from the inner surface of the cell membrane. For example, the tyrosine residue of the SLP-76 binding motif may be located approximately 238 amino acid residues from the inner surface of the cell membrane.

Consideration may also be given to the spacing of the GADS binding motif and SLP-76 binding motif with respect to one another, whether within a single polypeptide of a CAR of the invention, or in one or more proteins of a cell of the invention. Suitably, the tyrosine residue of the GADS binding motif and the tyrosine residue of the SLP-76 binding motif may be located between about 10 and 50 amino acid residues of one another. The tyrosine residue of the GADS binding motif and the tyrosine residue of the SLP-76 binding motif may be located between about 15 and 45 amino acid residues of one another. For example, the tyrosine residue of the GADS binding motif and the tyrosine residue of the SLP-76 binding motif may be located between about 20 and 40 amino acid residues of one another. Suitably, the tyrosine residue of the GADS binding motif and the tyrosine residue of the SLP-76 binding motif may be located between about 25 and 35 amino acid residues of one another, or between about 30 and 35 amino acid residues of one another. Suitably the tyrosine residue of the GADS binding motif and the tyrosine residue of the SLP-76 binding motif are located approximately 33 amino acid residues from one another.

In an embodiment in which the CD6-derived signalling region comprises a variant of the cytoplasmic region of CD6, or of a fragment of the cytoplasmic region of CD6, the variant may share at least 60% sequence identity with the corresponding native sequence (either the full length sequence of the cytoplasmic region or a fragment of this region). Suitably the variant may share at least 65% sequence identity, at least 70% sequence identity, at least 75% sequence identity, at least 80% sequence identity, at least 85% sequence identity, or at least 90% sequence identity with the corresponding native sequence. For example, the variant may share at least 91% sequence identity, at least 92% sequence identity, at least 93% sequence identity, at least 94% sequence identity, at least 95% sequence identity, at least 96% sequence identity, at least 97% sequence identity, at least 98% sequence identity, or at least 99% sequence identity with the corresponding fragment of the wild type CD6 protein.

It will be appreciated that the overall identity of a variant CD6-derived signalling region may be relatively low, as long as key areas, such as the GADS binding motif and SLP-76 binding motif, retain sufficiently high identity to allow the CD6-derived signalling region as a whole (and so the CAR or cell of the invention) to offer the increased capacity for T-cell activation that underpins the present invention.

Suitably a variant of the cytoplasmic region of CD6, or of a fragment of the cytoplasmic region of CD6, may differ from the corresponding native sequence by 2 or more amino acid residues, by 3 or more amino acid residues, by 4 or more amino acid residues, by 5 or more amino acid residues, by 6 or more amino acid residues, by 7 or more amino acid residues, by 8 or more amino acid residues, by 9 or more amino acid residues, or by 10 or more amino acid residues. A variant may differ from the corresponding native sequence by 15 or more amino acid residues, by 20 or more amino acid residues, by 25 or more amino acid residues, by 30 or more amino acid residues, by 35 or more amino acid residues, by 40 or more amino acid residues, by 45 or more amino acid residues, or by 50 or more amino acid residues.

It will be appreciated that in the case of CARs of the invention, embodiments in which the CD6-derived region comprises one or more fragment or variants of the native sequence of the CD6 cytoplasmic region, the CD6-derived region must still retain both a GADS binding motif and a SLP-76 binding motif. Fragments or variants that lose the capacity to bind GADS or SLP-76 will not fall within the scope of the present invention.

Similarly, in the case of cells of the invention, in which the CD6-derived region comprises one or more fragment or variants of the native sequence of the CD6 cytoplasmic region, whether on one or more proteins, the protein or proteins must jointly retain both a GADS binding motif and a SLP-76 binding motif. Fragments or variants that lose the capacity to bind GADS or SLP-76 will not fall within the scope of the invention.

### Antigen Binding Region

The antigen binding region of the CAR is a sequence presented on the surface of T-cells. They are engineered to have antigen binding specificity. This specificity enables the T-cell to target certain conditions or infections.

The antigen binding region may be a single chain variable fragment (scFv) scFv derived from immunoglobulins (Ig). The scFvs may be derived from murine Igs. scFv is a fusion protein of the variable regions of the heavy (V_{H}) and light V_{L}) chains of Immunoglobulins connected by a shorter linker peptide of about 10-25 amino acids. The antigen binding region may be a scFv against a cell surface antigen expressed on a tumour cell or virus containing cell.

Merely by way of example, the antigen binding region may be an scFv directed to CD19, an antigen commonly expressed by tumour cells. An example of such an scFv is incorporated in the CAR of the invention set out in SEQ ID NO: 13 (where the scFv close is made up of residues 1-285).

### Transmembrane Region

The transmembrane region of the CAR may be derived from T-cell protein molecules. Suitable T-cell molecules may be CD3 zeta, CD4, CD6, CD8 or CD28. Thus the transmembrane region may comprise transmembrane sequences from any of these T cell protein molecules. The transmembrane region may comprise CD28 with CD3 zeta. Such combination may result in high expression of CAR compared to using CD3 zeta alone.

A CAR or cell of the invention may comprise a transmembrane region from one, two, three, four or all five of these T cell protein molecules, in addition to the other regions specified herein.

A CAR or cell of the invention may combine a transmembrane region from CD8 with a cytoplasmic signalling region from CD3 zeta. The effectiveness of this combination is illustrated in the Examples.

Suitably, a CAR or cell of the invention may combine a transmembrane region, such as from any of the T cell protein molecules described above with a cytoplasmic signalling region from CD3 zeta. Indeed the CAR or cell of the invention may comprise a transmembrane region from CD6 or CD8 combined with both a cytoplasmic region from CD3 zeta and other CAR regions. By way of example a transmembrane region from CD6 or CD8 may be combined with a cytoplasmic region of CD3 zeta and with a cytoplasmic region of 4-1BB or cytoplasmic regions from both 4-1BB and CD28, as described further elsewhere in the specification.

It will be appreciated that the transmembrane region determines the location at which a CAR of the invention (or the relevant protein of a cell of the invention) is embedded in the cell membrane. Thus when calculating the distance of a part of such a CAR or protein (for example, the CD6 signalling region, the GADS binding motif or the SLP-76 binding motif) from the inner surface of the cell membrane, the distance from the transmembrane region can be used in circumstances in which the cell membrane is absent.

### Other CAR Regions

The CARs or cells of the invention may optionally comprise further regions, in addition to the antigen binding region, transmembrane region, and CD6-derived signalling regions described above.

The intracellular signalling region may comprise a signalling region of a CD3 zeta chain. The intracellular signalling region may comprise one or more signalling regions from CD28, ICOS, OX-40 or 4-1BB. The intracellular signalling region of a CAR or cell of the invention may comprise signalling regions from one, two, three, four or all five of these proteins in addition to the other regions specified herein.

The intracellular signalling region of a CAR or cell of the invention may comprise signalling regions from both 4-1BB and CD28. Suitably in such examples, the 4-1BB signalling region is downstream of the CD28 signalling region.

The CAR or cell of the invention may comprise a transmembrane region of CD8 and an intracellular region from CD3 zeta and a further intracellular region from 4-1BB or both 4-1BB and CD28.

The CAR may optionally comprise a spacer or hinge region situated between the antigen binding region and T cell plasma membrane. Commonly a spacer or hinge is a sequence derived from IgG subclass IgG1, IgG4, IgD or CD8.

A CAR may further comprise a linker region. This may be rich in glycine for flexibility. The linker region may be rich in serine and threonine for solubility. The linker region can connect to N-terminus of V_{H} with the C-terminus of the V_{L} or vice versa.

### Cells of the Invention

The second aspect of the invention provides cells that have been modified to express one or more proteins comprising regions of a CAR. When the one or more proteins are taken together, these regions jointly comprise an antigen binding region, a transmembrane region, a CD6-derived signalling region comprising a GADS binding motif and a CD6-derived signalling region comprising a SLP-76 binding motif.

The requisite regions of the CAR may be found on two, three, four or more proteins within the cells of the invention. Various permutations of these are described elsewhere in the specification.

The CD6-derived signalling region comprising the GADS binding motif and CD6-derived signalling region comprising the SLP-76 derived signalling region may both be provided in the same protein. Alternatively, the CD6-derived signalling region comprising the GADS binding motif and CD6-derived signalling region comprising the SLP-76 derived signalling region may be provided in two or more separate proteins.

This second aspect of the invention reflects the inventors' recognition that the advantages conferred by the CARs of the first aspect of the invention can also be gained in circumstances in which not all of the regions of the CARs of the first aspect of the invention are provided in a single protein.

The cell may be a T-cell. The cell may be selected from CD4+ and CD8+T-lymphocytes.

It will be appreciated that there are a number of different arrangements of proteins in which the requisite regions of the cells of the invention may be provided.

The regions of the CAR may be expressed as a single protein or as multiple proteins.

In one embodiment, the antigen binding region, transmembrane region, and both CD6-derived signalling regions may be expressed as a single protein. Such a single protein may optionally comprise further signalling regions, such as CD3 zeta.

In another embodiment, the antigen binding region and the transmembrane region may be expressed as a first protein and both the CD6-derived signalling regions may be expressed as a second protein. Further signalling regions, such as CD3 zeta, may be provided on the first and/or second protein.

In another embodiment, the antigen binding region and the transmembrane region may be expressed as a first protein, the CD6-derived signalling region comprising a GADS binding motif may be expressed as a second protein, and the CD6-derived signalling region comprising a SLP-76 binding motif may be expressed as a third protein.

In another embodiment, the antigen binding region, the transmembrane region, and the CD6-derived signalling region comprising a GADS binding motif may be expressed as a first protein, and the CD6-derived signalling region comprising a SLP-76 binding motif may be expressed as a second protein.

In another embodiment, the antigen binding region, the transmembrane region, and the CD6-derived signalling region comprising a SLP-76 binding motif may be expressed as a first protein, and the CD6-derived signalling region comprising a GADS binding motif may be expressed as a second protein.

In another embodiment, the antigen binding region, the transmembrane region, and the CD6-derived signalling region comprising a GADS binding motif may be expressed as a first protein, and the antigen binding region, the transmembrane region, and the CD6-derived signalling region comprising a SLP-76 binding motif may be expressed as a second protein.

The one or more nucleic acid sequences encoding the regions of the CAR, in accordance with the fourth aspect of the present invention, may be provided in one or more vectors.

Modification of the cell is by means of incorporation of one or more nucleic acids encoding the regions of the CAR into the cell such that the nucleic acids are capable of expression, specifically the incorporation of one or more nucleic acids encoding the antigen binding region, the transmembrane region and the CD6-derived signalling regions.

### Nucleic Acids and Vectors

The third and fourth aspects of the invention respectively relate to nucleic acids encoding the CARs of the first aspect of the invention or the cells of the second aspect of the invention. The nucleic acids of the invention may be DNA or RNA. Suitably the nucleic acids of the invention may be combinations of DNA and RNA, or derivatives of these DNA or RNA.

An exemplary nucleic acid encoding the exemplary chimeric receptor (of SEQ ID NO. 4) is set out in SEQ ID NO. 7. An exemplary nucleic acid encoding a CAR of the invention (of SEQ ID NO: 13) is set out in SEQ ID NO: 11.

The sequence of DNA encoding human CD6 is set out in SEQ ID NO. 5, and the sequence of DNA encoding the CD6-derived signalling region of SEQ ID NO. 3 is set out in SEQ ID NO. 6.

It will be appreciated that codon degeneracy means that nucleic acids encoding the same protein may significantly differ from one another in terms of identity. Furthermore, the ability to employ variants of the naturally occurring CD6 sequence in the CD6-derived signalling regions of the CARs or cells of the invention allows the use of nucleic acids that further vary from the naturally occurring nucleic acid sequences or exemplary sequence set out herein.

Accordingly, in a suitable embodiment, a nucleic acid sequence of the third aspect of the invention may comprise a nucleic acid sequence sharing at least 50% sequence identity with the portion of SEQ ID NO. 1 or SEQ ID NO. 7 or SEQ ID NO: 11 encoding a corresponding region of the protein. Suitably such a nucleic acid may share at least 60% sequence identity, at least 70% sequence identity, at least 80% sequence identity, or at least 90% sequence identity with the corresponding portion of SEQ ID NO. 1 or SEQ ID NO. 7 or SEQ ID NO: 11.

By the same token, the collection of nucleic acid sequences of the fourth aspect of the invention, encoding proteins of the second aspect of the invention may comprise a nucleic acid sequence sharing at least 50% sequence identity with the portion of SEQ ID NO. 1 or SEQ ID NO. 7 or SEQ ID NO: 11 encoding a corresponding region of the protein. Suitably such a nucleic acid may share at least 60% sequence identity, at least 70% sequence identity, at least 80% sequence identity, or at least 90% sequence identity with the corresponding portion of SEQ ID NO. 1 or SEQ ID NO. 7 or SEQ ID NO: 11.

As for the CARs or cells of the invention before, it will be appreciated that the overall identity of a nucleic acid encoding a CD6-derived signalling may be relatively low, as long those parts of the sequence encoding key areas, such as the GADS binding motif and SLP-76 binding motif retain sufficiently high identity to encode proteins that offer the increased capacity for T-cell activation that underpins the present invention.

The nucleic acids of the third aspect of the invention, or the collections of nucleic acids may be provided to a cell by any suitable means known to those skilled in the art. Suitable means may be selected with reference to the cell type, or to the form of the nucleic acids, be they DNA or RNA.

The nucleic acids of the third aspect of the invention, or collections of nucleic acids of the fourth aspect of the invention, may be provided incorporated in one or more expression vectors, to facilitate their cellular expression. The vector may be a viral vector. For example, the vector may be selected from a lentiviral vector and a retroviral vector. The vector may be one suitable for stable integration in the cell.

Use of an expression vector to incorporate one or more nucleic acids encoding the regions of the CAR into the cell constitutes a suitable method by which CARS of the first aspect of the invention, or cells of the second aspect of the invention, may be produced.

### Increased T-Cell Activation

The CARs of the first aspect of the invention are able to dramatically increase the activation of T-cells into which they are incorporated. Similarly, T-cells in accordance with the second aspect of the invention may demonstrate the same marked increase in activation. This increase in T-cell activation is illustrated by the experimental chimeric receptor, and experimental CARs and cells of the invention, discussed further in the examples.

T-cell activation may be assessed experimentally in a number of different ways. Merely by way of example, IL-2 production by cells of the invention, or cells expressing CARs of the invention, is a suitable readout for T cell activation. IL-2 production in CAR T cells in response to stimulation has been shown to correlate with tumour cell killing in vitro and survival of mice in mouse tumour models. Further details of a method by which IL-2 production by cells of the invention, or cells expressing CARs of the invention, can be assessed are set out in the Examples.

Other readouts for T cell activation may be selected from the group consisting of: the production of other cytokines (i.e. cytokines other than IL-2) such as interferon-gamma; and increased T cell proliferation. As set out in the examples, exemplary cells of the invention expressing CARs of the invention demonstrate a significantly increase in interferon-gamma expression when activated (in this case in response to cancer cells), as compared to controls. Thus increased interferon-gamma expression may represent a particularly useful indication of increased T-cell activation in the context of the CARs or cells of the invention.

In addition, T cells can be incubated with tumour cells and specific lysis of tumour cells can be measured. Similarly, T cell activation may also be indicated by exocytosis of cytotoxic granules (which may have the ability to cause specific cell lysis). Thus an increase in exocytotic cytotoxic granule release and/or an increase in target cell lysis provides a suitable indication of increased T cell activation on the part of a T cell of the invention or a cell incorporating the CAR of the invention. Measurements of T cell activation by exocytosis of cytotoxic granules, and by the ability of T cells to cause specific cell lysis, are set out in the Examples. The Examples clearly illustrate that the CARs and cells of the invention offer benefits in terms of increased cytotoxic granule exocytosis, and improved target cell killing, even in examples where other indications of activation do not appear to be elevated. Since killing of target cells underpins the clinical use of CARs and cells of the invention, as discussed further below, then it will be appreciated that CARs and cells of the invention demonstrating this improvement may be clinically useful even if they do not demonstrate increases in other indications of activation.

It is known that studies using cells of hybridoma lines exhibit lower levels of T-cell activation than do primary T-cells. Nevertheless, the inventors have demonstrated a threefold increase in efficacy using a hybridoma model. The benefits of the CARs and cells of the invention have also been demonstrated in primary T cells, as set out further in the Examples.

Suitably the cells of the invention, or cells expressing CARs of the invention, may achieve at least a threefold, at least a fourfold, at least a fivefold, or greater increase in T-cell activation as compared to suitable comparator cells (e.g. cells lacking the CD6-derived signalling region). Suitably the cells of the invention, or cells expressing CARs of the invention, may achieve at least a sixfold, at least a sevenfold, at least an eightfold, at least a ninefold, or at least a tenfold or greater increase in T-cell activation. Cells of the invention, or cells expressing CARs of the invention, may achieve at least a 20-fold, at least a 30-fold, at least a 40-fold, or at least a 50-fold, or greater, increase in T-cell activation as compared to suitable comparator cells.

It will be appreciated that it is T-cell activation that confers the therapeutic utility of these cells in applications such as the prevention or treatment of cancer, or treatment of infections. Merely by way of example, cytotoxic granules are secreted during recognition of target cells, such as tumour cells, by activated T cells, and the release of these results in tumour cell lysis. Accordingly, the increased activation that may be achieved by T-cells in accordance with the second aspect of the invention, or CARs of the first aspect of the invention incorporated in T-cells, means that these cells are well placed to be employed in medical uses, or in methods of treatment, as described herein.

### Medical Uses of the CARs or Cells of the Invention, and Methods of Treatment Utilising the CARs or Cells

According to the fifth aspect of the invention, there is provided a method of treating a condition in a subject in need thereof, the method comprising providing the subject with a CAR in accordance with the first aspect of the invention. It will be appreciated that this fifth aspect of the invention also provides the CAR of the first aspect of the invention for use as a medicament. As described further herein, the CAR of the first aspect of the invention may be used in the treatment of cancer. Alternatively, the CAR of the first aspect of the invention may be used in the treatment of viral infections.

According to the sixth aspect of the invention, there is provided a method of treating a condition in a subject in need thereof, the method comprising providing the subject with a cell in accordance with the second aspect of the invention. It will also be appreciated that this sixth aspect of the invention also provides the cells of the second aspect of the invention for use as a medicament. As described further herein, the cells of the second aspect of the invention may be used in the treatment of cancer. Alternatively, the cells of the second aspect of the invention may be used in the treatment of viral infections.

The methods of treatment of the fifth and sixth aspects of the invention are of particular utility in the treatment of cancer, though they may also be used in treatment of other conditions.

Merely by way of example, the methods of treatment of the fifth and sixth aspects of the invention may be used to kill virus-infected cells. As in tumour therapy, stronger T cell activation increases the killing of virus-infected cells. Therefore, the increased T cell activation observed in respect of the CARs and cells of the invention is likely to lend itself to therapy of viral infections.

A method in accordance with the fifth or sixth aspect of the invention may be used in the treatment of blood cancers, and in particular of B-cell leukaemia. In this disease the tumour cells express the surface molecule CD19. The methods of the fifth or sixth aspects of the invention may be of particular benefit in the treatment of B-cell acute lymphoblastic leukaemia, chronic lymphocytic leukaemia or B-cell non-Hodgkin lymphoma (Park, Geyer and Brentjens. CD19 CARs clinical outcomes. Blood 2016).

The methods of the fifth or sixth aspect of the invention may be used in the treatment of solid tumours, since the stronger activation that they are able to achieve may be helpful in overcoming the immunosuppressive environment of solid tumours.

The methods of treatment of the fifth and sixth aspects of the invention refer to providing a subject requiring treatment respectively with either a CAR or cell in accordance with the invention. It will be appreciated that the CAR or cell can be provided directly (by administration of the cell, or a cell expressing the CAR, directly to the subject), or indirectly (for example by administration of a nucleic acid of the third aspect of the invention, or collection of nucleic acids of the fourth aspect of the invention, to the subject).

Merely by way of example, in embodiments in which cells of the invention, or comprising CARs of the invention, are to be administered directly to a subject, a suitable (for example, in the treatment of CD19 positive leukaemias), a dose of between 1×10⁶ and 11×10⁸ cells may be used. A suitable dose may be increased as appropriate in the context of treatment of solid tumours.

In a suitable embodiment for practicing the methods of the fifth or sixth aspect of the invention, T cells from a subject cells may be isolated from the patient, and then activated, for example using beads coated with antibodies such as anti-CD3 and anti-CD28 antibodies. The activated cells may then be transduced, for example with a vector, such as a viral vector. The cells may be incubated with a suitable vector for a period sufficient to allow their transduction and expansion of cell numbers. The cells may then be transferred back into the patient.

Alternatively, the cells may be isolated, and transduced with a nucleic acid, or collection of nucleic acids, encoding the CAR. The nucleic acid, or nucleic acids, may, for example, be used in an expression vector. The cells may then be activated and expanded, for example using beads coated with anti-CD3 and anti-CD28 antibodies. The cells may then be transferred back into the patient.

The invention will now be further described with reference to the following Examples and accompanying Figures.

### EXAMPLES

### Study 1

1 Role of CD6 Y629 and Y662 in Jurkat activation after CD3 and CD6 antibody stimulation.

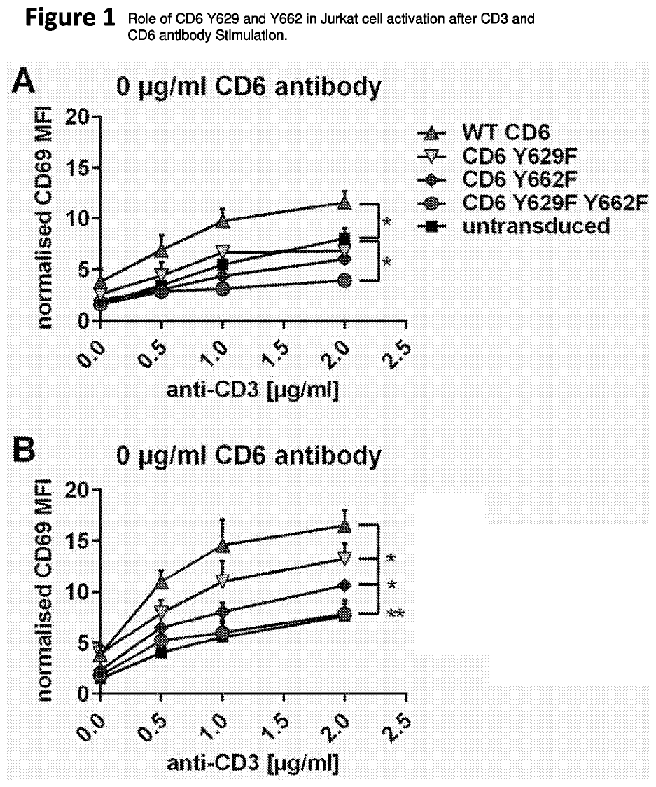

The inventors investigated the roles of tyrosine Y629 (subsequently identified as part of the GADS binding motif) and tyrosine Y662 (the key residue of the SLP-76 binding motif) in activation of Jurkat cells after stimulation with antibodies to either CD3 or CD6. Cells were transduced to express mutant forms of CD6 in which one or both of these tyrosine residues had been substituted. The results of this study are shown in FIG. 1.

In FIG. 1(A) Jurkat cells were stimulated with CD3 antibodies for 18 h and the CD69 mean florescence intensity (MFI) was examined for WT CD6, CD6 Y629F, CD6 Y662F or CD6 Y629F Y662F transduced cells or untransduced cells. Statistics are shown for comparisons of WT or mutant CD6 transduced cells with untransduced cells. The study reported in FIG. 1(B) was fundamentally the same as for panel A, but cells were also treated with 2.5 μg/ml CD6 antibody. CD69 MFI for VVT CD6 was significantly higher (p<0.01) than for untransduced cells for any CD3 concentration. Statistics are shown for comparisons of mutant CD6 transduced cells with VVT CD6 transduced cells. n=3, error bars show the standard error of the mean, * p<0.05, ** p<0.01 student's t test for each CD3 antibody concentration above 0 μg/ml.

2 Co-immunoprecipitation of CD6 mutants shows a cooperation of CD6 Y629 and Y662

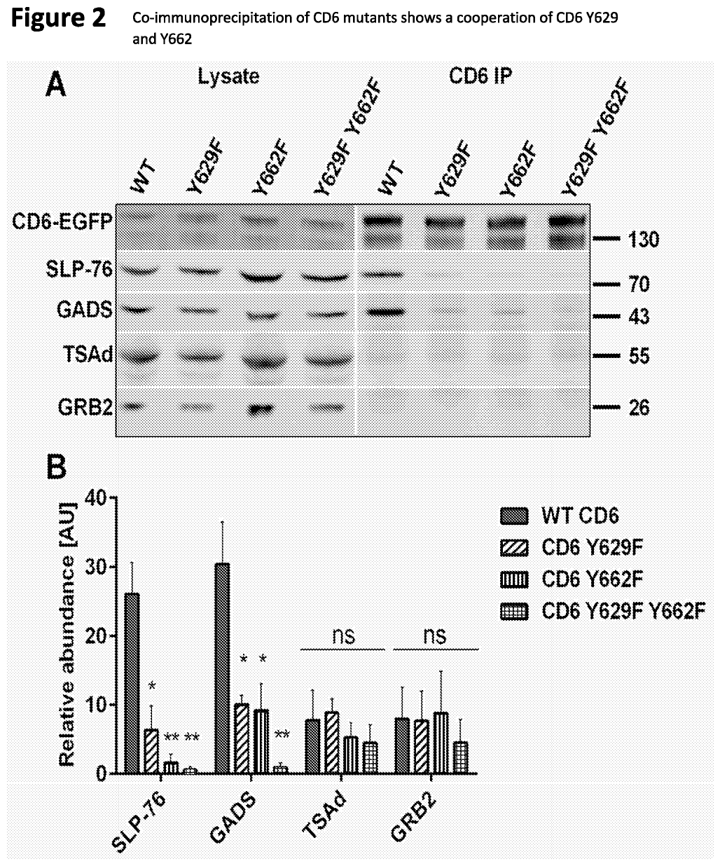

The inventors investigated the relevance of Y629 and Y662 to CD6's binding of GADS and SLP-76. Their results, set out in FIG. 2 and discussed further below, illustrate that binding of GADS and SLP-76 to CD6 is dependent on both tyrosine residues. CD6 Y629 and Y662 alone can only recruit a small amount of GADS and SLP-76 and both residues are required for efficient recruitment of GADS and SLP-76.

In FIG. 2(A) Lysate of 8×10⁵ Jurkat cells treated with the phosphatase inhibitor Na₃VO₄ or immunoprecipitation samples of 3×10⁶ cells were loaded on gels and after electrophoretic separation they were blotted on nitrocellulose membranes. The membranes were probed with the indicated antibodies and visualised using LI-COR western blotting. FIG. 2(B) illustrates Western Blot bands of three independent CD6 co-immunoprecipitations were quantified using the software ImageJ. Each co-immunoprecipitation value is divided by the intensity of the band in the lysate to compensate for differences in cell numbers used and is normalised to the intensity of EGFP in the co-immunoprecipitation divided by the intensity of EGFP in the lysate to compensate for different expression levels of the constructs. Samples were analysed using the unpaired Student t test comparing values of mutants to the VVT. n=3, error bars show the standard error of the mean, * p<0.05, ** p<0.01, ns=not significant.

Blotting for EGFP shows similar levels of CD6-EGFP in the lysates and a similar enrichment for the different CD6 constructs in the immunoprecipitation. SLP-76 and GADS were co-immunoprecipitated with WT CD6 but the co-immunoprecipitation of SLP-76 and GADS was strongly reduced with CD6 Y629F and CD6 Y662F mutants. Mutating CD6 Y629 and Y662 further reduced the signal and almost no SLP-76 and GADS was detected in the co-immunoprecipitation sample.

3 the Activating Effect of CD6 in Primary T Cells Assessed by CD69 Expression is Mediated by the Y629 and Y662 Residues

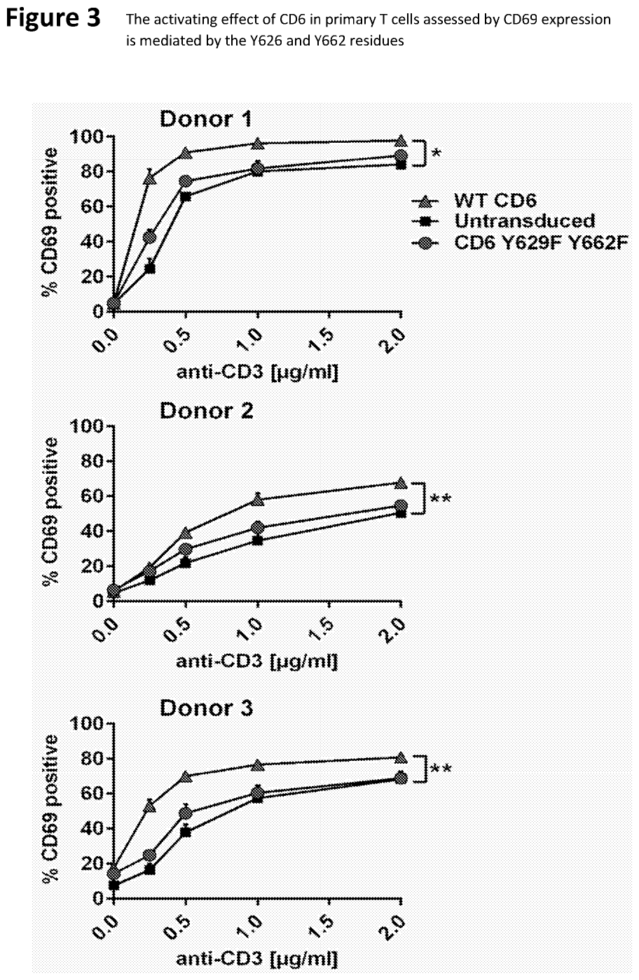

The inventors investigated the roles of Y629 and Y662 in activation of primary T cells. The results of this study are set out in FIG. 3.

CD4+ T cells were stimulated with CD3 antibodies and 5 μg/ml CD6 antibodies to crosslink transduced CD6. WT CD6 transduced T cells of all three donors had a significantly increased percentage of CD69 positive cells, confirming the activating effect of crosslinking CD6 together with crosslinking CD3 seen in Jurkat cells. The costimulatory function of VVT CD6 was seen with different CD3 antibody concentrations but not in the absence of CD3 antibodies, suggesting that CD6 ligation alone cannot activate T cells.

Mutating the CD6 tyrosine residues Y629 and Y662 significantly reduced the T cell response compared to VVT CD6 transduced T cells. In fact, there was no significant difference between untransduced cells and CD6 Y629F Y662F transduced cells. The difference between WT CD6 and Y629F Y662F CD6 or untransduced cells was increased at 0.25 μg/ml CD3 antibody for donor 1 and 3 compared to higher CD3 antibody concentrations, suggesting that at weaker TCR stimulation, CD6 has a stronger costimulatory effect. These results indicate that the CD6 Y629 and Y662 residues are essential for CD6-mediated costimulation.

4 The activating effect of CD6 on IL-2 production in primary T cells is mediated by the CD6 Y629 and Y662 residues.

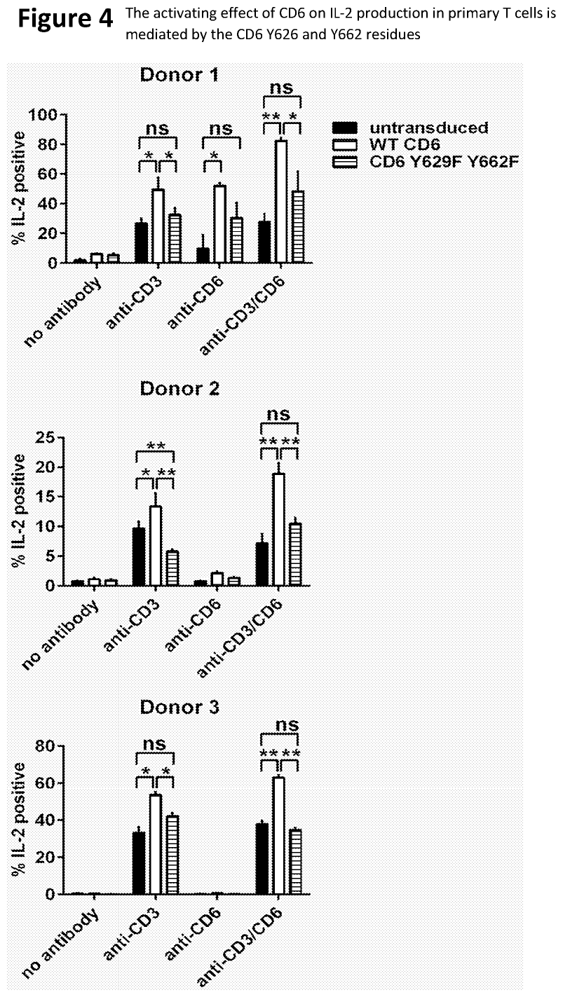

The inventors investigated the role of the Y629 (GADS binding motif) and Y662 (SLP-76 binding motif) residues in CD6's ability to activate IL-2 production in primary T cells. The results of this study are set out in FIG. 4.

When T cells were stimulated with CD3 antibodies but not CD6 antibodies, T cells transduced with WT CD6 had a significantly increased percentage of IL-2 positive cells. T cells expressing CD6 Y629F Y662F (mutated tyrosine residues) did not show a significantly increased level of T cell activation and in donor 2, there is evidence that CD6 Y629F Y662F can inhibit the percentage of IL-2 positive cells.

Stimulation of T cells with both CD3 antibodies and CD6 antibodies significantly increased the response of VVT CD6 transduced T cells for all three donors. T cells transduced with CD6 Y629F Y662F did not show a significantly increased percentage of IL-2 positive cells.

T cells were stimulated with 2 μg/ml CD3 antibody and 5 μg/ml CD6 antibody for 18 h and assessed for the percentage of CD69 positive cells. In FIG. 4, n=3, and error bars show the standard error of the mean. “*” denotes p<0.05, “**” denotes p<0.01, and “ns” denotes that differences were not significant, when assessed by student's t test.

Thus, results obtained with intracellular IL-2 as readout for T cell activation confirm the CD69 data for primary T cells and Jurkat cells. This means that it is likely that the effects of VVT CD6 and CD6 Y629F Y662F are not specific to a particular readout but are real influences on T cell activation.

5 Incorporation of GADS Binding Motif and SLP-76 Binding Motif from CD6 into Chimeric Receptors Increases T-Cell Activation

The inventors produced an exemplary chimeric receptor comprising the intracellular regions of a CAR in accordance with the invention. The sequence of this exemplary chimeric receptor is set out in SEQ ID NO. 4.

The activity of the exemplary chimeric receptor was compared with that of a comparator chimeric receptor lacking the CD6-derived signalling region. Both chimeric receptor molecules contained the extracellular and transmembrane regions of human CD6 and the cytoplasmic region of mouse CD3 zeta. The exemplary chimeric receptor further comprised residues 623 to 668 from the C terminus of human CD6.

Murine T hybridoma cells were transduced to express either the exemplary or comparator chimeric receptors. Cells were assessed for their response to human CD6 monoclonal antibody immobilised on 96 well tissue culture plates. After incubation for 18 h at 37° C., the supernatants were analysed for the presence of mouse IL-2 by ELISA.

### CD6 Exemplary Chimeric Receptor Test

The activity of a comparator chimeric receptor (consisting of the extracellular region of human CD6 and the mouse CD3 zeta tail, effectively a first generation CAR in the cytoplasmic region) was investigated alongside the exemplary chimeric receptor illustrative of CARs or cells of the invention. This exemplary receptor shared the same sequence as the comparator, but further comprised the human CD6 C terminus (starting with residue 622 to the stop codon, 47 amino acids in length) added to the C terminus of the CD3 zeta chain. These comparator and exemplary chimeric receptors were transduced into 2B4 Reay cells, a mouse T cell hybridoma line. The cells were stimulated with different concentrations of plate-bound T12.1 monoclonal antibody (mAb), a CD6 crosslinking antibody, overnight and secreted mouse IL-2 was measured by ELISA.

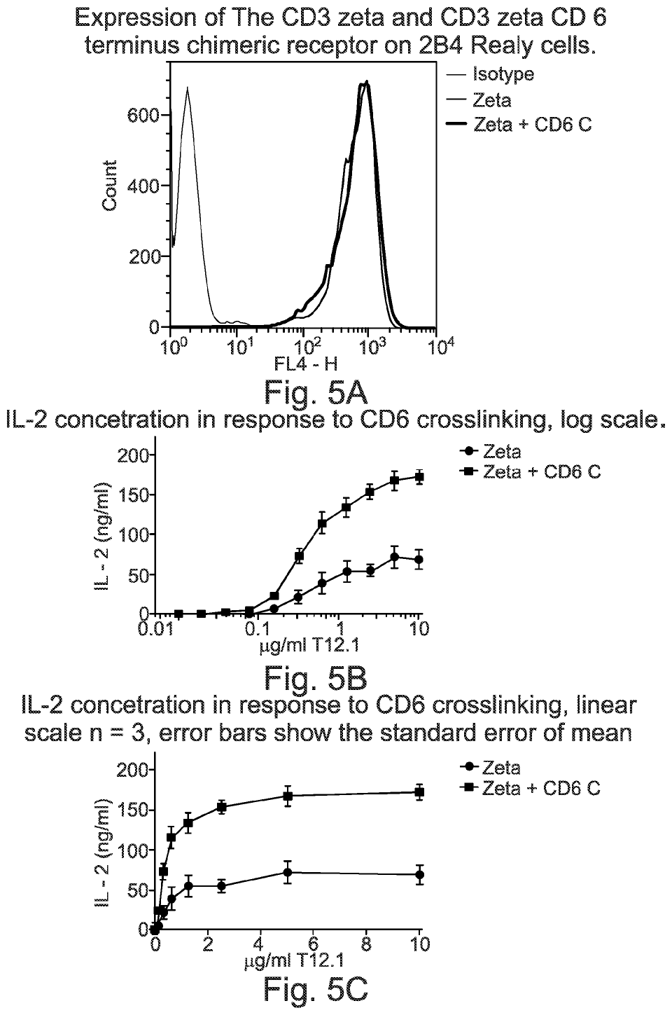

The results of this study are shown in FIG. 5. The comparator chimeric receptor is labelled “Zeta”, and the exemplary chimeric receptor is labelled “Zeta+CD6”.

FIG. 5(A) illustrates expression of the “Zeta” comparator and “Zeta+CD6” exemplary chimeric receptor (so designated due to the presence of the C terminus of CD6) in 2B4 Reay cells.

FIG. 5(B) illustrates IL-2 concentration (on a log scale) produced by cells expressing comparator chimeric receptors or exemplary chimeric receptors in response to CD6 crosslinking. FIG. 5(C) illustrates IL-2 concentration by the same cells in response to CD6 crosslinking on a linear scale. n=3, error bars show the standard error of mean.

6 GADS SH2 domain specifically binds to CD6 pY629 and that the SLP-76 SH2 domain specifically binds to CD6 pY662.

The recombinant GADS SH2 and SLP-76 SH2 domains were expressed in bacteria as his-tagged proteins and purified according to their nickel affinity and size. On a streptavidin-coated CM5 chip, biotinylated CD6 peptides containing the Y629 and the Y662 residues with either or both tyrosine residues phosphorylated were immobilised. In a BIAcore 3000, equilibrium binding constants of the SH2 domains were measured at 15° C. and 37° C. 37° C. was chosen to measure specificity at a physiological temperature and 15° C. was chosen to determine if cross-specificity is revealed at lower temperatures.

The GADS SH2 domain bound to the CD6 pY629 Y662 and the CD6 pY629 pY662 peptide with similar affinities but it did not bind to the CD6 Y629 pY662 peptide at 15° C. and 37° C. The SLP-76 SH2 domain bound to the CD6 Y629 pY662 and the CD6 pY629 pY662 peptide with similar affinities but it did not bind to the CD6 pY629 Y662 peptide at 15° C. and 37° C. The equilibrium dissociation constants measured for the SLP-76 SH2 domain for CD6 pY662 are similar to published measurements.

These results are shown in Table 1 below, and prove that the GADS SH2 domain specifically binds to CD6 pY629 and that the SLP-76 SH2 domain specifically binds to CD6 pY662.

TABLE 1
K_{D} for GADS SH2 and SLP-76 SH2 for pY629 and pY662

| | CD6 peptide | GADS SH2 | SLP-76 SH2 |
|---|---|---|---|
| 15° C. | pY629 Y662 | 0.15-0.62* | — |
| | Y629 pY662 | — | 0.31-1.87 |
| | pY629 pY662 | 0.14-2.05 | 0.26-1.82 |
| 37° C. | pY629 Y662 | 0.60-0.88 | — |
| | Y629 pY662 | — | 0.89-3.09 |
| | pY629 pY662 | 0.77-1.58 | 0.62-2.82 |

*KD given in μM

### Study 2

### Materials and Methods

### CAR Constructs

The pHR SIN-BX-IRES-Emerald expression vector was used for CAR construct transduction of T cells. The CAR constructs comprised:
an extracellular region consisting of a single chain variable regions binding to human CD19
a transmembrane region derived from human CD8
a cytoplasmic region that consisted of the human CD3 zeta chain.

Details of a suitable anti-CD19 CAR that may be modified to produce a CAR of the invention are set out in Milone, et al. 2009.

Additional costimulatory regions tested in combination with the CAR constituents above were derived from the cytoplasmic regions of the receptors 4-1BB and CD28. The costimulatory region of 4-1BB was tested either alone, or in combination with CD28 (in which case the region from 4-1BB was placed downstream of CD28).

The constructs above were tested with or without incorporation of the human CD6 C terminus (sequence starting with residue 622 and continuing to the stop codon—47 amino acids in length) at the C terminus of the construct. It will be appreciated that those constructs that incorporated the CD6-derived signalling region constitute experimental examples of CARs or cells of the invention, whilst those without the CD6-derived region provided suitable controls. The various experimental CARs produced are labelled 4-1BB zeta, with or without CD6, in the accompanying Figures.

### CAR T Cell Production

Human embryonic kidney cells (HEK 293T cells) were transfected with CAR constructs previously described for 20 hours. CD4+ and CD8+ T cells were isolated from human blood using a RosetteSep enrichment cocktail (Stemcell Technologies) and Ficoll (GE Healthcare). The T cells were activated for 20 hours with 25 ρl/1×10⁶ cells Dynabeads human T-activator CD3/CD28 and 100 U/ml human IL-2 (Life Technologies). The viral supernatants produced by the transfected HEK 293T cells were added to the activated T cells for transduction, producing CAR T cells expressing the constructs previously described. All assays were conducted 10-14 days after T cell activation.

### 4-1BB Zeta and 4-1BB Zeta CD6 CARs of the Invention are Expressed at the Same Levels on CD4+ and CD8+ T Cells

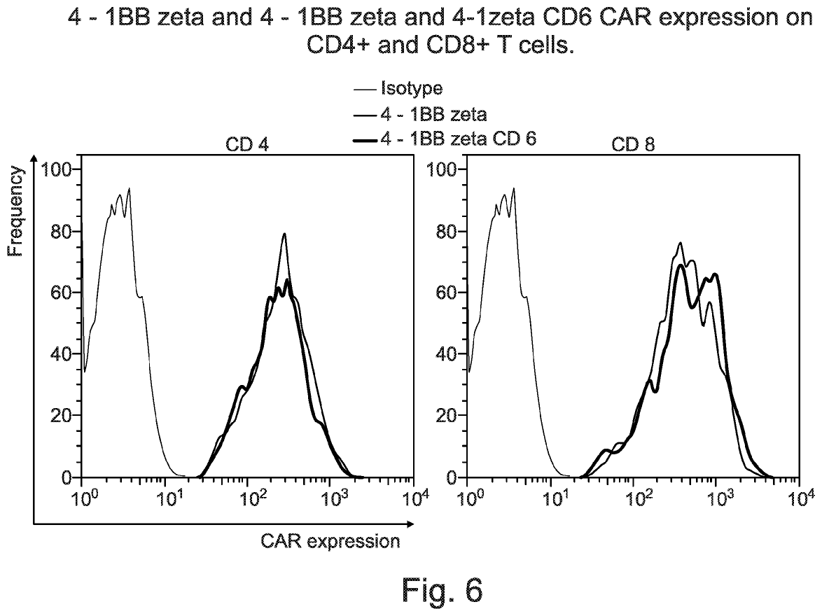

Detection of CAR expression was carried out as follows. CAR T cells were stained with 15 μg/ml Fab-specific goat anti-mouse IgG biotin (Sigma-Aldrich) and 1 μg/ml streptavidin-APC (BD Pharmingen) and analysed by flow cytometry. The results are shown in FIG. 6. On CD4+ and CD8+ T cells, both CARs are expressed at similar levels and therefore, adding the CD6-derived signalling region does not affect 4-1BB zeta CAR expression.

### Cytokine Production by Activated CD4+ CAR T Cells of the Invention

Cytokine secretion is a measure of T cell activation and therefore a suitable readout for CAR function. CAR T cells were incubated overnight with varying amounts of the CD19+ Burkitt lymphoma cell line, Daudi. The CD19 expressed by the Daudi cells is recognised by the anti-CD19 moiety in the extracellular region of the cells of the invention and control cells.

CD4 T cells transduced with CARs of the invention or control CARs (as described above) were incubated with Daudi target cells overnight. Production of cytokines by the T cells in response to incubation with the target cells was then assessed. Secretion of the cytokines interleukin-2 (IL-2) and interferon gamma (IFNγ) by the cells was measured by ELISA.

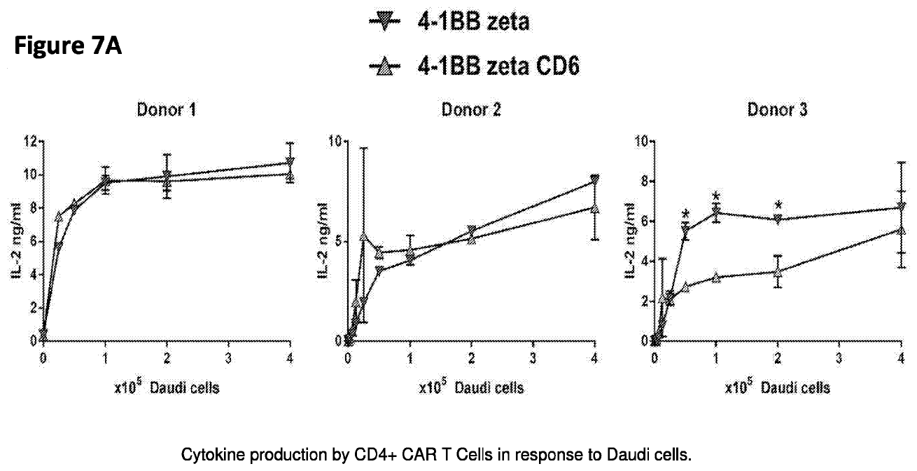

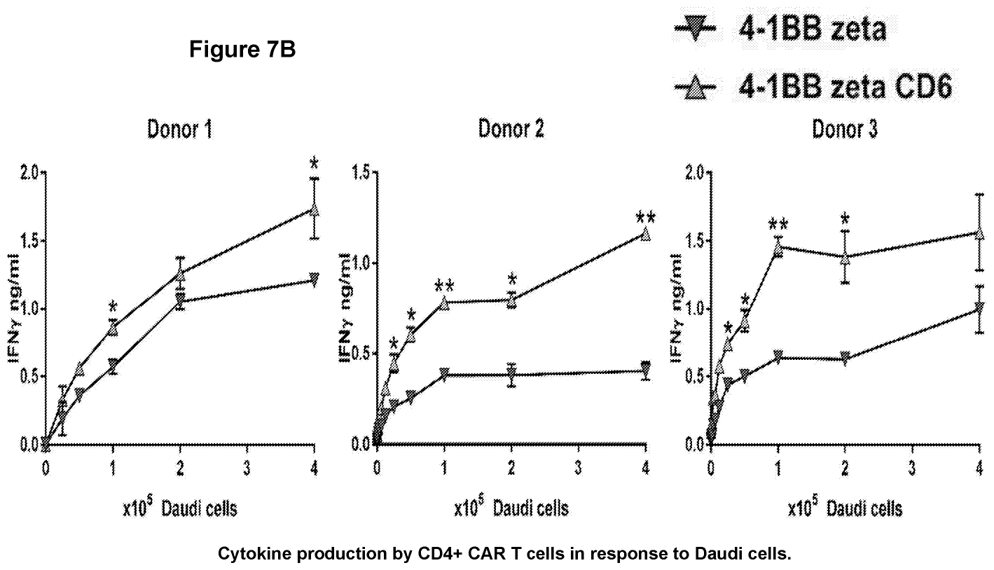

The results are shown in FIG. 7A and FIG. 7B, which respectively set out the values for IL-2 and IFNγ secretion measured by ELISA. The results shown are from two independent experiments with duplicates in each experiment. *p<0.05, **p<0.01 Student's t test for the indicated amounts of Daudi cells.

The cytokines IL-2 and IFNγ were measured as a readout of T cell activation (FIG. 7). The results obtained illustrated a difference between expression of the two cytokines in response to exposure to target cells. There was no consistent difference in secreted IL-2 between the 4-1BB zeta CAR transduced T cells and 4-1BB zeta CD6 CAR transduced T cells (FIG. 7A). However, IFNγ production by 4-1BB zeta CD6 cells was increased (as compared to controls) on exposure to larger numbers of target cells. This increase compared to control cells was statistically significant when the cells were incubated with 10⁵ or 4×10⁵ Daudi cells (FIG. 7B). Furthermore, the inventors found that incubation of the CD4+ CAR T cells of the invention with CD19− Jurkat cells did not result in cytokine secretion. This indicates that the CARs of the invention are specific for target cells expressing the CD19 antigen.

IFNγ has important anti-tumour activity in vivo. Accordingly, the increased expression of this cytokine by the CAR-T cells of the invention indicates that they are likely to have improved ability to kill target cells, such as tumour cells, as compared to control cells. The skilled reader will appreciate that CAR-T cells incorporating cytoplasmic regions of 4-1BB and CD3 zeta have previously been shown to have many beneficial properties (such as desirable proliferation), However, their capacity to kill tumour cells is not elevated, when compared with other types of CAR. The finding that CARs of the invention incorporating cytoplasmic domains of 4-1BB have improved IFNγ secretion and cancer cell killing capabilities (discussed further below), as compared to control 4-1BB CAR-T cells, indicates that these CARs and cells of the invention may be able to retain the beneficial properties of 4-1BB CAR-T cells while adding a desirably increased ability to kill target cells. Indeed, the inventors believe that the killing capacity of the cells of the invention is elevated to levels that have not previously been observed.

The results demonstrate that adding the CD6 C terminus to a 4-1BB zeta CAR does not result in any change in IL-2 secretion compared to control CARs suggesting that the addition of the CD6 C terminus does not have any adverse impact upon the biological activity. Cytokine secretion generally correlates with cell proliferation, and so these results indicate that the desirable proliferation properties of the starting CAR-T cells are retained in the CAR-T cells of the invention.

### Cytotoxic Granule Exocytosis by CD8+ CAR T Cells

The CAR T cells used were produced by the methods described above.

Cytotoxic granule exocytosis is required for efficient target cell killing and occurs during recognition of target cells such as tumour cells and therefore indicate the cytotoxic function of CD8+ T cells. A marker for cytotoxic granules is CD107a (LAMP-1) which will migrate to the cell surface during exocytosis. 5×10⁴ CD8+ CAR T cells from donors 1-3 were incubated for 5 hours with varying amounts of Daudi target cells in the presence of a CD107a mAb labelled with the fluorophore APC (Miltenyi Biotec) and 2 μM Monensin (Biolegend). Monensin prevents acidification of phagosomes and therefore prevents quenching of internalised fluorophores during internalisation of CD107a after exocytosis. The signal of the CD107a antibody was measured by flow cytometry. Results shown are from two independent experiments with duplicates in each experiment.*p<0.05, **p<0.01, Student's t test.

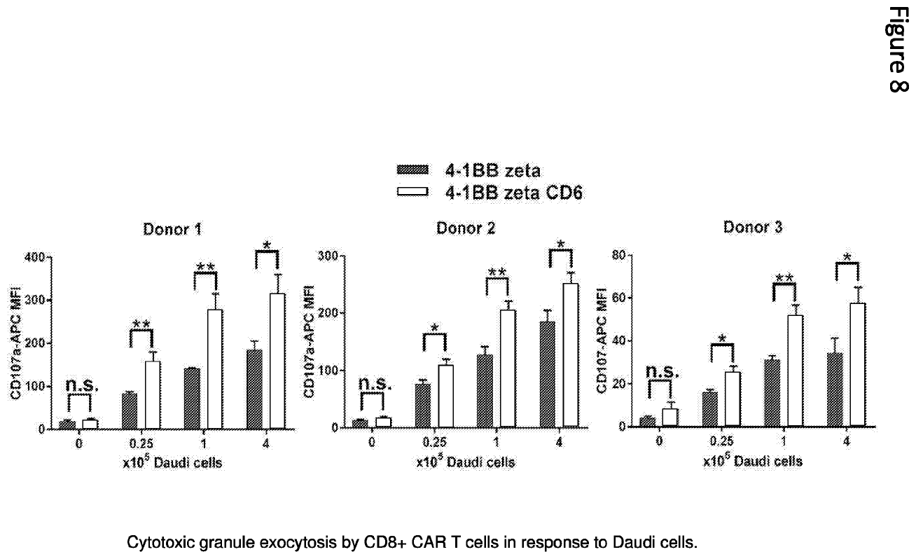

For all Daudi amounts, the 4-1BB zeta CD6 CAR T cells of the invention resulted in a significantly increased CD107a signal as compared with 4-1BB zeta control CAR T cells (FIG. 8). These results indicate stronger exocytosis of cytotoxic granules in the 4-1BB zeta CD6 CAR T cells. This suggests that the CD6 C terminus significantly enhanced granule exocytosis of the 4-1BB zeta CAR consistent with increased killing of tumour cells by CAR T cells. The inventors found that incubation of the CD8+ CAR T cells of the invention with CD19-Jurkat cells did not lead to cytotoxic granule exocytosis. This provides a further indication that the CARs of the invention are specific for target cells expressing the CD19 antigen.

### Tumour Cell Killing by CD8+ CAR T Cells

The CAR T cells used were produced by the methods described above. Cytotoxicity assays provide a direct indication of the capability of CAR T cells to kill cancer cells by cell lysis.

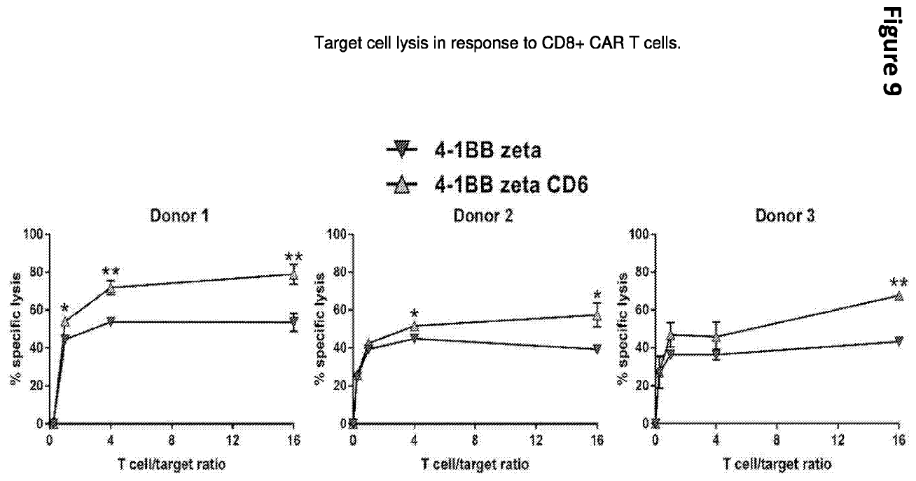

CD19+ Daudi cells (target cells) were labelled with 10 μM of the cell dye CFSE and CD19-Jurkat T cell leukaemia cells (a negative control) were labelled with 1 μM CFSE. Daudi and Jurkat cells were then mixed in a 1:1 ratio and incubated for 6 hours with varying amounts of CD8+ CAR T cells. The ratio of Daudi cells to Jurkat cells was measured by flow cytometry. As the results (reported elsewhere in the Examples) demonstrate that the CAR T cells of the invention do not cause any non-specific killing of the CD19− Jurkat control cells, the change in ratio of Daudi to Jurkat cells was taken as evidence of specific lysis of the CD19+ Daudi cells. (FIG. 9).

The results demonstrate that adding the CD6 C terminus to 4-1BB zeta CAR T cells, resulted in significant enhanced tumour cell killing by up to 32% for both donors at a T cell/target ratio of 16:1. This confirmed that the increased exocytosis of cytotoxic granules led to increased lysis of tumour cells. Results shown are from two independent experiments with duplicates in each experiment. *p<0.05, **p<0.01, Student's t test for the indicated ratios of T cells to target cells.

### Adding the CD6 C Terminus to a 4-1BB Zeta CAR Enhances Lysis of Tumour Cells Measured by the Release of Lactate Dehydrogenase

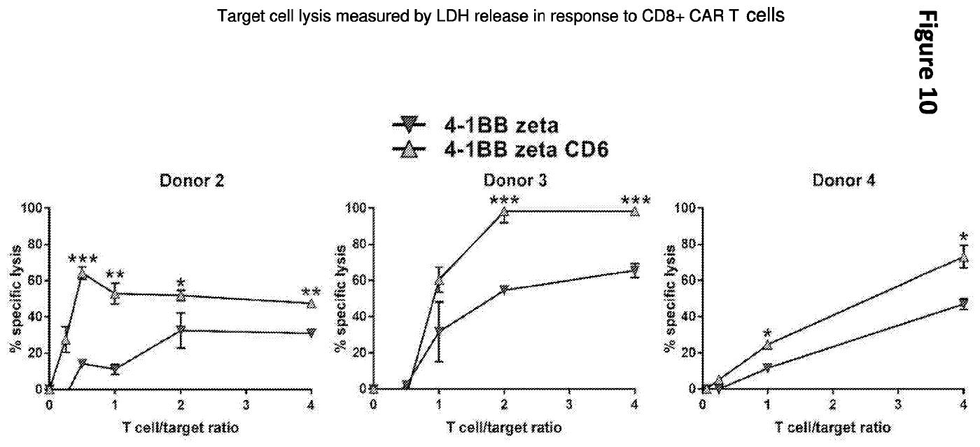

Dying cells release the enzyme lactate dehydrogenase (LDH) which can be measured by examining LDH enzymatic function in the culture medium. 0.4×10⁵ Daudi cells were incubated with varying amounts of CD8+ CAR T cells for 6 hours. The release of LDH was measured using the CytoTox 96 non-radioactive cytotoxicity assay (Promega) as indication of Daudi cell lysis according to the manufacturer's protocol. The results (in FIG. 10) show that adding CD6 to a 4-1BB zeta CAR significantly increases target cell lysis, confirming the flow cytometry killing assay.

SEQUENCE INFORMATION
Human CD6
SEQ ID NO. 1
MWLFFGITGLLTAALSGHPSPAPPDQLNTSSAESELWEPGERLPVRLTNGSSSCSGTVEV
RLEASWEPACGALWDSRAAEAVCRALGCGGAEAASQLAPPTPELPPPPAAGNTSVAANAT
LAGAPALLCSGAEWRLCEVVEHACRSDGRRARVTCAENRALRLVDGGGACAGRVEMLEH
GEWGSVCDDTWDLEDAHVVCRQLGCGWAVQALPGLHFTPGRGPIHRDQVNCSGAEAYL
WDCPGLPGQHYCGHKEDAGAVCSEHQSWRLTGGADRCEGQVEVHFRGVWNTVCDSEW
YPSEAKVLCQSLGCGTAVERPKGLPHSLSGRMYYSCNGEELTLSNCSWRFNNSNLCSQSL
AARVLCSASRSLHNLSTPEVPASVQTVTIESSVTVKIENKESRELMLLIPSIVLGILLLGSLIFIA
FILLRIKGKYALPVMVNHQHLPTTIPAGSNSYQPVPITIPKEVFMLPIQVQAPPPEDSDS
GSDSDYEHYDFSAQPPVALTTFYNSQRHRVTDEEVQQSRFQMPPLEEGLEELHASHIPTA
NPGHCITDPPSLGPQYHPRSNSESSTSSGEDYCNSPKSKLPPWNPQVFSSERSSFLEQPP
NLELAGTQPAFSAGPPADDSSSTSSGEWYQNFQPPPQPPSEEQFGCPGSPSPQPDSTDN
DDYDDISAA
Cytoplasmic region of human CD6
SEQ ID NO. 2
RIKGKYALPVMVNHQHLPTTIPAGSNSYQPVPITIPKEVFMLPIQVQAPPPEDSDSGSD
SDYEHYDFSAQPPVALTTFYNSQRHRVTDEEVQQSRFQMPPLEEGLEELHASHIPTANPG
HCITDPPSLGPQYHPRSNSESSTSSGEDYCNSPKSKLPPWNPQVFSSERSSFLEQPPNLE
LAGTQPAFSAGPPADDSSSTSSGEWYQNFQPPPQPPSEEQFGCPGSPSPQPDSTDNDDY
DDISAA
CD6-derived signalling region suitable for incorporation in a CAR
of the invention
SEQ ID NO. 3
TSSGEWYQNFQPPPQPPSEEQFGCPGSPSPQPDSTDNDDYDDISAA
Exemplary murine/human chimeric receptor
SEQ ID NO. 4
MWLFFGITGLLTAALSGHPSPAPPDQLNTSSAESELWEPGERLPVRLTNGSSSCSGTVEVR
LEASWEPACGALWDSRAAEAVCRALGCGGAEAASQLAPPTPELPPPPAAGNTSVAANATL
AGAPALLCSGAEWRLCEVVEHACRSDGRRARVTCAENRALRLVDGGGACAGRVEMLEHG
EWGSVCDDTWDLEDAHVVCRQLGCGWAVQALPGLHFTPGRGPIHRDQVNCSGAEAYLW
DCPGLPGQHYCGHKEDAGAVCSEHQSWRLTGGADRCEGQVEVHFRGVWNTVCDSEWY
PSEAKVLCQSLGCGTAVERPKGLPHSLSGRMYYSCNGEELTLSNCSWRFNNSNLCSQSLA
ARVLCSASRSLHNLSTPEVPASVQTVTIESSVTVKIENKESRELMLLIPSIVLGILLLGSLIFIAFI
LRAKFSRSAETAANLQDPNQLYNELNLGRREEYDVLEKKRARDPEMGGKQQRRRNPQE
GVYNALQKDKMAEAYSEIGTKGERRRGKGHDGLYQGLSTATKDTYDALHMQTLAPRT
SSGEWYQNFQPPPQPPSEEQFGCPGSPSPQPDSTDNDDYDDISAA
No text style (until underlined text): human CD6 extracellular region
Underlined: human CD6 transmembrane region
Bold: mouse CD3 zeta cytoplasmic region
Italic: human CD6 signalling region
DNA encoding human CD6
SEQ ID NO. 5
ATGTGGCTCTTCTTCGGGATCACTGGATTGCTGACGGCAGCCCTCTCAGGTCATCCAT
CTCCAGCCCCACCTGACCAGCTCAACACCAGCAGTGCAGAGAGTGAGCTCTGGGAGC
CAGGGGAGCGGCTTCCGGTCCGTCTGACAAACGGGAGCAGCAGCTGCAGCGGGACG
GTGGAGGTGCGGCTCGAGGCGTCCTGGGAGCCCGCGTGCGGGGCGCTCTGGGACAG
CCGCGCCGCCGAGGCCGTGTGCCGAGCACTGGGCTGCGGCGGGGCGGAGGCCGCC
TCTCAGCTCGCCCCGCCGACCCCTGAGCTGCCGCCCCCGCCTGCAGCCGGGAACACC
AGCGTAGCAGCTAATGCCACTCTGGCCGGGGCGCCCGCCCTCCTGTGCAGCGGCGCC
GAGTGGCGGCTCTGCGAGGTGGTGGAGCACGCGTGCCGCAGCGACGGGAGGCGGGC
CCGTGTCACCTGTGCAGAGAACCGCGCGCTGCGCCTGGTGGACGGTGGCGGCGCCT
GCGCCGGCCGCGTGGAGATGCTGGAGCATGGCGAGTGGGGATCAGTGTGCGATGAC
ACTTGGGACCTGGAGGACGCCCACGTGGTGTGCAGGCAACTGGGCTGCGGCTGGGCA
GTCCAGGCCCTGCCCGGCTTGCACTTCACGCCCGGCCGCGGGCCTATCCACCGGGAC
CAGGTGAACTGCTCGGGGGCCGAAGCTTACCTGTGGGACTGCCCGGGGCTGCCAGGA
CAGCACTACTGCGGCCACAAAGAGGACGCGGGCGCGGTGTGCTCAGAGCACCAGTCC
TGGCGCCTGACAGGGGGCGCTGACCGCTGCGAGGGGCAGGTGGAGGTACACTTCCG
AGGGGTCTGGAACACAGTGTGTGACAGTGAGTGGTACCCATCGGAGGCCAAGGTGCT
CTGCCAGTCCTTGGGCTGTGGAACTGCGGTTGAGAGGCCCAAGGGGCTGCCCCACTC
CTTGTCCGGCAGGATGTACTACTCATGCAATGGGGAGGAGCTCACCCTCTCCAACTGC
TCCTGGCGGTTCAACAACTCCAACCTCTGCAGCCAGTCGCTGGCAGCCAGGGTCCTCT
GCTCAGCTTCCCGGAGTTTGCACAATCTGTCCACTCCCGAAGTCCCTGCAAGTGTTCA
GACAGTCACTATAGAATCTTCTGTGACAGTGAAAATAGAGAACAAGGAATCTCGGGAGC
TAATGCTCCTCATCCCCTCCATCGTTCTGGGAATTCTCCTCCTTGGCTCCCTCATCTTCA
TAGCCTTCATCCTCTTGAGAATTAAAGGAAAATATGCCCTCCCCGTAATGGTGAACCAC
CAGCACCTACCCACCACCATCCCGGCAGGGAGCAATAGCTATCAACCGGTCCCCATCA
CCATCCCCAAAGAAGTTTTCATGCTGCCCATCCAGGTCCAGGCCCCGCCCCCTGAGGA
CTCAGACTCTGGCTCGGACTCAGACTATGAGCACTATGACTTCAGCGCCCAGCCTCCT
GTGGCCCTGACCACCTTCTACAATTCCCAGCGGCATCGGGTCACAGATGAGGAGGTCC
AGCAAAGCAGGTTCCAGATGCCACCCTTGGAGGAAGGACTTGAAGAGTTGCATGCCTC
CCACATCCCAACTGCCAACCCTGGACACTGCATTACAGACCCGCCATCCCTGGGCCCT
CAGTATCACCCGAGGAGCAACAGTGAGTCGAGCACCTCTTCAGGGGAGGATTACTGCA
ATAGTCCCAAAAGCAAGCTGCCTCCATGGAACCCCCAGGTGTTTTCTTCAGAGAGGAG
TTCCTTCCTGGAGCAGCCCCCAAACTTGGAGCTGGCCGGCACCCAGCCAGCCTTTTCA
GCAGGGCCCCCGGCTGATGACAGCTCCAGCACCTCATCCGGGGAGTGGTACCAGAAC
TTCCAGCCACCACCCCAGCCCCCTTCGGAGGAGCAGTTTGGCTGTCCAGGGTCCCCC
AGCCCTCAGCCTGACTCCACCGACAACGATGACTACGATGACATCAGCGCAGCCTAG
Ensembl accession number: ENST00000313421
DNA encoding the CD6-derived signalling region of SEQ ID NO. 3
SEQ ID NO. 6
ACCTCATCCGGGGAGTGGTACCAGAACTTCCAGCCACCACCCCAGCCCCCTTCGGAG
GAGCAGTTTGGCTGTCCAGGGTCCCCCAGCCCTCAGCCTGACTCCACCGACAACGATG
ACTACGATGACATCAGCGCAGCC
DNA encoding the exemplary chimeric receptor of SEQ ID NO. 4
SEQ ID NO. 7
ATGTGGCTCTTCTTCGGGATCACTGGATTGCTGACGGCAGCCCTCTCAGGTCATCCAT
CTCCAGCCCCACCTGACCAGCTCAACACCAGCAGTGCAGAGAGTGAGCTCTGGGAGC
CAGGGGAGCGGCTTCCGGTCCGTCTGACAAACGGGAGCAGCAGCTGCAGCGGGACG
GTGGAGGTGCGGCTCGAGGCGTCCTGGGAGCCCGCGTGCGGGGCGCTCTGGGACAG
CCGCGCCGCCGAGGCCGTGTGCCGAGCACTGGGCTGCGGCGGGGCGGAGGCCGCC
TCTCAGCTCGCCCCGCCGACCCCTGAGCTGCCGCCCCCGCCTGCAGCCGGGAACACC
AGCGTAGCAGCTAATGCCACTCTGGCCGGGGCGCCCGCCCTCCTGTGCAGCGGCGCC
GAGTGGCGGCTCTGCGAGGTGGTGGAGCACGCGTGCCGCAGCGACGGGAGGCGGGC
CCGTGTCACCTGTGCAGAGAACCGCGCGCTGCGCCTGGTGGACGGTGGCGGCGCCT
GCGCCGGCCGCGTGGAGATGCTGGAGCATGGCGAGTGGGGATCAGTGTGCGATGAC
ACTTGGGACCTGGAGGACGCCCACGTGGTGTGCAGGCAACTGGGCTGCGGCTGGGCA
GTCCAGGCCCTGCCCGGCTTGCACTTCACGCCCGGCCGCGGGCCTATCCACCGGGAC
CAGGTGAACTGCTCGGGGGCCGAAGCTTACCTGTGGGACTGCCCGGGGCTGCCAGGA
CAGCACTACTGCGGCCACAAAGAGGACGCGGGCGCGGTGTGCTCAGAGCACCAGTCC
TGGCGCCTGACAGGGGGCGCTGACCGCTGCGAGGGGCAGGTGGAGGTACACTTCCG
AGGGGTCTGGAACACAGTGTGTGACAGTGAGTGGTACCCATCGGAGGCCAAGGTGCT
CTGCCAGTCCTTGGGCTGTGGAACTGCGGTTGAGAGGCCCAAGGGGCTGCCCCACTC
CTTGTCCGGCAGGATGTACTACTCATGCAATGGGGAGGAGCTCACCCTCTCCAACTGC
TCCTGGCGGTTCAACAACTCCAACCTCTGCAGCCAGTCGCTGGCAGCCAGGGTCCTCT
GCTCAGCTTCCCGGAGTTTGCACAATCTGTCCACTCCCGAAGTCCCTGCAAGTGTTCA
GACAGTCACTATAGAATCTTCTGTGACAGTGAAAATAGAGAACAAGGAATCTCGGGAGC
TAATGCTCCTCATCCCCTCCATCGTTCTGGGAATTCTCCTCCTTGGCTCCCTCATCTTCA
TAGCCTTCATCCTCTTGAGAGCAAAATTCAGCAGGAGTGCAGAGACTGCTGCCAACC
TGCAGGACCCCAACCAGCTCTACAATGAGCTCAATCTAGGGCGAAGAGAGGAATATG
ACGTCTTGGAGAAGAAGCGGGCTCGGGATCCAGAGATGGGAGGCAAACAGCAGAG
GAGGAGGAACCCCCAGGAAGGCGTATACAATGCACTGCAGAAAGACAAGATGGCAG
AAGCCTACAGTGAGATCGGCACAAAAGGCGAGAGGCGGAGAGGCAAGGGGCACGA
TGGCCTTTACCAGGGTCTCAGCACTGCCACCAAGGACACCTATGATGCCCTGCATAT
GCAGACCCTGGCCCCTCGCACCTCATCCGGGGAGTGGTACCAGAACTTCCAGCCACC
ACCCCAGCCCCCTTCGGAGGAGCAGTTTGGCTGTCCAGGGTCCCCCAGCCCTCAGCC
TGACTCCACCGACAACGATGACTACGATGACATCAGCGCAGCC
No text style (until underlined text): human CD6 extracellular region
Underlined: human CD6 transmembrane region
Bold: mouse CD3 zeta cytoplasmic region
Italic: human CD6 signalling region
GADS binding motif
SEQ ID NO. 8
YQNF
SLP-76 binding motif
SEQ ID NO. 9
YDDI
Anti-CD19 scFv 4-1BB zeta CAR
SEQ ID NO: 10
ATGGAGACCGACACCCTGCTGCTGTGGGTGCTGCTGCTGTGGGTGCCAGGCAGCACC
GGCGACATCCAGATGACCCAGACCACCAGCAGCCTGAGCGCCAGCCTGGGCGACCGG
GTGACCATCAGCTGCAGAGCCAGCCAGGACATCAGCAAGTACCTGAACTGGTACCAGC
AGAAGCCCGACGGCACCGTGAAGCTGCTGATCTACCACACCAGCCGGCTGCACAGCG
GCGTGCCCAGCCGGTTCAGCGGCAGCGGCAGCGGCACCGACTACAGCCTGACCATCA
GCAACCTGGAGCAGGAGGACATCGCCACCTACTTCTGCCAGCAGGGCAACACCCTGC
CCTACACCTTCGGAGGCGGCACCAAGCTGGAGATCACCAAGGCCGGAGGCGGAGGCT
CTGGCGGAGGCGGCTCTGGCGGAGGCGGCTCTGGCGGAGGCGGCAGCGAGGTGAA
GCTGCAGGAGTCTGGCCCAGGCCTGGTGGCCCCAAGCCAGAGCCTGAGCGTGACCTG
CACCGTGAGCGGCGTGAGCCTGCCCGACTACGGCGTGAGCTGGATCAGGCAGCCCCC
ACGGAAGGGCCTGGAGTGGCTGGGCGTGATCTGGGGCAGCGAGACCACCTACTACAA
CAGCGCCCTGAAGAGCCGGCTGACCATCATCAAGGACAACAGCAAGAGCCAGGTGTT
CCTGAAGATGAACAGCCTGCAGACCGACGACACCGCCATCTACTACTGCGCCAAGCAC
TACTACTATGGCGGCAGCTACGCTATGGACTACTGGGGCCAGGGCACCAGCGTGACC
GTGAGCTCGGATCCCACCACCACCCCAGCCCCACGGCCACCTACCCCTGCCCCAACC
ATCGCCAGCCAGCCCCTGAGCCTGCGGCCTGAAGCCTGCAGGCCTGCCGCCGGAGG
AGCCGTGCACACAAGGGGCCTGGACTTCGCCTGCGACATCTATATCTGGGCCCCCCTG
GCCGGGACATGCGGGGTGCTGCTGCTGTCCCTGGTGATTACACTGTATTGCAAACGG
GGCCGGAAGAAGCTGCTGTACATCTTCAAGCAGCCCTTCATGCGGCCCGTGCAGACC
ACCCAGGAGGAGGACGGCTGCAGCTGCCGGTTCCCCGAGGAAGAGGAAGGCGGCT
GCGAGCTGCGGGTGAAGTTCAGCCGGAGCGCCGACGCCCCAGCCTACCAGCAGGGC
CAGAACCAGCTGTACAACGAGCTGAACCTGGGACGGCGGGAGGAGTACGACGTGCTG
GACAAGCGGCGGGGACGGGACCCCGAGATGGGCGGCAAGCCTCGCCGGAAGAATCC
CCAGGAGGGCCTGTACAACGAGCTGCAGAAGGACAAGATGGCCGAGGCCTACAGCGA
GATCGGCATGAAGGGCGAGCGGCGCCGGGGCAAGGGCCACGACGGCCTGTACCAGG
GCCTGAGCACCGCCACCAAGGACACCTACGACGCCCTGCACATGCAGGCCCTGCCAC
CCCGGTGA
Anti-CD19 scFv 4-IBB zeta CD6 CAR of the invention
SEQ ID NO: 11
ATGGAGACCGACACCCTGCTGCTGTGGGTGCTGCTGCTGTGGGTGCCAGGCAGCACC
GGCGACATCCAGATGACCCAGACCACCAGCAGCCTGAGCGCCAGCCTGGGCGACCGG
GTGACCATCAGCTGCAGAGCCAGCCAGGACATCAGCAAGTACCTGAACTGGTACCAGC
AGAAGCCCGACGGCACCGTGAAGCTGCTGATCTACCACACCAGCCGGCTGCACAGCG
GCGTGCCCAGCCGGTTCAGCGGCAGCGGCAGCGGCACCGACTACAGCCTGACCATCA
GCAACCTGGAGCAGGAGGACATCGCCACCTACTTCTGCCAGCAGGGCAACACCCTGC
CCTACACCTTCGGAGGCGGCACCAAGCTGGAGATCACCAAGGCCGGAGGCGGAGGCT
CTGGCGGAGGCGGCTCTGGCGGAGGCGGCTCTGGCGGAGGCGGCAGCGAGGTGAA
GCTGCAGGAGTCTGGCCCAGGCCTGGTGGCCCCAAGCCAGAGCCTGAGCGTGACCTG
CACCGTGAGCGGCGTGAGCCTGCCCGACTACGGCGTGAGCTGGATCAGGCAGCCCCC
ACGGAAGGGCCTGGAGTGGCTGGGCGTGATCTGGGGCAGCGAGACCACCTACTACAA
CAGCGCCCTGAAGAGCCGGCTGACCATCATCAAGGACAACAGCAAGAGCCAGGTGTT
CCTGAAGATGAACAGCCTGCAGACCGACGACACCGCCATCTACTACTGCGCCAAGCAC
TACTACTATGGCGGCAGCTACGCTATGGACTACTGGGGCCAGGGCACCAGCGTGACC
GTGAGCTCGGATCCCACCACCACCCCAGCCCCACGGCCACCTACCCCTGCCCCAACC
ATCGCCAGCCAGCCCCTGAGCCTGCGGCCTGAAGCCTGCAGGCCTGCCGCCGGAGG
AGCCGTGCACACAAGGGGCCTGGACTTCGCCTGCGACATCTATATCTGGGCCCCCCTG
GCCGGGACATGCGGGGTGCTGCTGCTGTCCCTGGTGATTACACTGTATTGCAAACGG
GGCCGGAAGAAGCTGCTGTACATCTTCAAGCAGCCCTTCATGCGGCCCGTGCAGACC
ACCCAGGAGGAGGACGGCTGCAGCTGCCGGTTCCCCGAGGAAGAGGAAGGCGGCT
GCGAGCTGCGGGTGAAGTTCAGCCGGAGCGCCGACGCCCCAGCCTACCAGCAGGGC
CAGAACCAGCTGTACAACGAGCTGAACCTGGGACGGCGGGAGGAGTACGACGTGCTG
GACAAGCGGCGGGGACGGGACCCCGAGATGGGCGGCAAGCCTCGCCGGAAGAATCC
CCAGGAGGGCCTGTACAACGAGCTGCAGAAGGACAAGATGGCCGAGGCCTACAGCGA
GATCGGCATGAAGGGCGAGCGGCGCCGGGGCAAGGGCCACGACGGCCTGTACCAGG
GCCTGAGCACCGCCACCAAGGACACCTACGACGCCCTGCACATGCAGGCCCTGCCAC
CCCGG

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

Anti-CD19 scFv 4-1BB zeta CAR
SEQ ID NO: 12
METDTLLLWVLLLWVPGSTGDIQMTQTTSSLSASLGDRVTISCRASQDISKYLNWYQQKPD
GTVKLLIYHTSRLHSGVPSRFSGSGSGTDYSLTISNLEQEDIATYFCQQGNTLPYTFGGGTK
LEITKAGGGGSGGGGSGGGGSGGGGSEVKLQESGPGLVAPSQSLSVTCTVSGVSLPDYG
VSWIRQPPRKGLEWLGVIWGSETTYYNSALKSRLTIIKDNSKSQVFLKMNSLQTDDTAIYYC
AKHYYYGGSYAMDYWGQGTSVTVSSDPTTTPAPRPPTPAPTIASQPLSLRPEACRPAAGG
AVHTRGLDFACDIYIWAPLAGTCGVLLLSLVITLYCKRGRKKLLYIFKQPFMRPVQTTQEED
GCSCRFPEEEEGGCELRVKFSRSADAPAYQQGQNQLYNELNLGRREEYDVLDKRRGRDP
EMGGKPRRKNPQEGLYNELQKDKMAEAYSEIGMKGERRRGKGHDGLYQGLSTATKDTYD
ALHMQALPPR*
Anti-CD19 scFv 4-1BB zeta CD6 CAR of the invention
SEQ ID NO: 13
METDTLLLWVLLLWVPGSTGDIQMTQTTSSLSASLGDRVTISCRASQDISKYLNWYQQKPD
GTVKLLIYHTSRLHSGVPSRFSGSGSGTDYSLTISNLEQEDIATYFCQQGNTLPYTFGGGTK
LEITKAGGGGSGGGGSGGGGSGGGGSEVKLQESGPGLVAPSQSLSVTCTVSGVSLPDYG
VSWIRQPPRKGLEWLGVIWGSETTYYNSALKSRLTIIKDNSKSQVFLKMNSLQTDDTAIYYC
AKHYYYGGSYAMDYWGQGTSVTVSSDPTTTPAPRPPTPAPTIASQPLSLRPEACRPAAGG
AVHTRGLDFACDIYIWAPLAGTCGVLLLSLVITLYCKRGRKKLLYIFKQPFMRPVQTTQEED
GCSCRFPEEEEGGCELRVKFSRSADAPAYQQGQNQLYNELNLGRREEYDVLDKRRGRDP
EMGGKPRRKNPQEGLYNELQKDKMAEAYSEIGMKGERRRGKGHDGLYQGLSTATKDTYD
ALHMQALPPR [symbol] [symbol] [symbol] [symbol] [symbol] [symbol]
In SEQ ID NOs: 10-13:
Anti-CD19 scFv (non-bold, non-underlined)
CD8 transmembrane region (non-bold, underlined)
4-1BB (bold, underlined)
Zeta (italicised)

[symbol]

[symbol]

[symbol]

[symbol]

1. A chimeric antigen receptor (CAR) comprising an antigen binding region, a transmembrane region and a CD6-derived signalling region, wherein the CD6-derived signalling region comprises a GADS binding motif and a SLP-76 binding motif.

2. The CAR according to claim 1, wherein the CD6-derived signalling region is between 20 and 60 amino acid residues in length.

3. The CAR according to claim 1 or claim 2, wherein the CD6-derived signalling region is between 170 and 230 amino acid residues from the inner surface of a cell membrane.

4. The CAR according to any preceding claim, wherein the GADS binding motif comprises at least a tyrosine residue.

5. The CAR according to claim 4, wherein the tyrosine residue corresponds to amino acid 629 of SEQ ID NO.1.

6. The CAR according to claim 4 or claim 5, wherein the GADS binding motif comprises amino acid residues YXN.

7. The CAR according to claim 6, wherein the GADS binding motif comprises amino acid residues YQNF.

8. The CAR according to any preceding claim, wherein the GADS binding motif is located between 180 and 230 amino acids from the inner surface of the cell membrane.

9. The CAR according to any preceding claim, wherein the SLP-76 binding motif comprises at least a tyrosine residue.

10. The CAR according claim 8, wherein the tyrosine residue corresponds to amino acid 662 of SEQ ID NO.1.

11. The CAR according to claim 8 or claim 9, wherein the SLP-76 binding motif comprises amino acid residues YXD.

12. The CAR according to claim 10 or 11, wherein the SLP-76 binding motif comprises amino acid residues YDDI.

13. The CAR according to any preceding claim, wherein the SLP-76 binding motif is located between 205 and 265 amino acids from the inner surface of the cell membrane.

14. The CAR according to any preceding claim, wherein the GADS binding motif and the SLP-76 binding motif are within the same fragment of the native sequence of the CD6-derived signalling region.

15. The CAR according to any preceding claim, wherein the GADS binding motif and the SLP-76 binding motif are within different fragments of the native sequence of the CD6-derived signalling region.

16. The CAR according to any preceding claim, wherein the GADS binding motif and the SLP-76 binding motif are located between 20 and 40 amino acids of one another.

17. The CAR according to any preceding claim, wherein the CD6-derived signalling region comprises the amino acid sequence of SEQ ID NO.3.

18. The CAR according to any preceding claim, wherein the CD6-derived signalling region consists of the amino acid sequence of SEQ ID NO.3.

19. The CAR according to any of claims 1 to 16, wherein the CD6-derived signalling region is a variant sharing at least 60% sequence identity with a native CD6-derived signalling region.

20. A CAR according to any preceding claim sharing at least 90% identity with the amino acid sequence of SEQ ID NO: 13.

21. A CAR according to claim 20, comprising the amino acid sequence of SEQ ID NO: 13.

22. A CAR according to claim 21, consisting of the amino acid sequence of SEQ ID NO: 13.

23. A nucleic acid sequence encoding a CAR according to any preceding claim.

24. A method of treating a condition in a subject in need thereof, the method comprising providing the subject with a CAR according to any of claims 1 to 22.

25. The method according to claim 24, wherein the CAR is provided in a cell modified to express the CAR according to any of claims 1 to 22.

26. The method according to claim 24 or claim 25, wherein the CAR is provided by cellular expression of a nucleic acid sequence according to claim 23.

27. The method according to any of claims 24 to 26 in the treatment of cancer.

28. The method according to any of claims 24 to 26 in the treatment of viral infection.

29. A cell modified to express one or more proteins comprising regions of a chimeric antigen receptor (CAR), said regions jointly comprising:
an antigen binding region;
a transmembrane region;
a CD6-derived signalling region comprising a GADS binding motif; and
a CD6-derived signalling region comprising a SLP-76 binding motif.

30. The cell according to claim 29, wherein the CD6-derived signalling region comprising the GADS binding motif and the CD6-derived signalling region comprising the SLP-76 derived signalling region are both provided in the same protein.

31. The cell according to claim 29, wherein the CD6-derived signalling region comprising the GADS binding motif and the CD6-derived signalling region comprising the SLP-76 derived signalling region are provided in two or more separate proteins.

32. The cell according to any of claims 29 to 31, wherein the CD6-derived signalling region is between 20 and 60 amino acid residues in length.

33. The cell according to any of claims 29 to 32, wherein the CD6-derived signalling region is between 170 and 230 amino acid residues from the inner surface of a cell membrane.

34. The cell according to any of claims 29 to 33, wherein the GADS binding motif comprises at least a tyrosine residue.

35. The cell according to claim 34, wherein the tyrosine residue corresponds to Y629 of SEQ ID NO.1.

36. The cell according to claim 34 or claim 35, wherein the GADS binding motif comprises amino acid residues YXN.

37. The cell according to claim 36, wherein the GADS binding motif comprises amino acid residues YQNF.

38. The cell according to any of claims 29 to 37, wherein the GADS binding motif is located between 180 and 230 amino acids from the inner surface of the cell membrane.

39. The cell according to any of claims 29 to 38, wherein the SLP-76 binding motif comprises at least a tyrosine residue.

40. The cell according claim 39, wherein the tyrosine residue corresponds to Y662 of SEQ ID NO.1.

41. The cell according to claim 39 or claim 40, wherein the SLP-76 binding motif comprises amino acid residues YXD.

42. The cell according to claim 41, wherein the SLP-76 binding motif comprises amino acid residues YDDI.

43. The cell according to any of claims 29 to 42, wherein the SLP-76 binding motif is located between 205 and 265 amino acids from the inner surface of the cell membrane.

44. The cell according to any of claim 29 to claim 43, wherein the GADS binding motif and the SLP-76 binding motif are within the same fragment of the native sequence of the CD6-derived signalling region or variant thereof.

45. The cell according to any of claim 29 to claim 44, wherein the GADS binding motif and the SLP-76 binding motif are within different fragments of the native sequence of the CD6-derived signalling region or variant thereof.

46. The cell according to any of claims 29 to 45, wherein the GADS binding motif and the SLP-76 binding motif are located between 20 and 40 amino acids of one another.

47. The cell according to any of claims 29 to 46, wherein the CD6-derived signalling region comprises the amino acid sequence of SEQ ID NO.3.

48. The cell according to any of claims 29 to 47, wherein the CD6-derived signalling region consists of the amino acid sequence of SEQ ID NO.3.

49. The cell according to any of claims 29 to 46, wherein the CD6-derived signalling region is a variant sharing at least 60% sequence identity with native CD6-derived signalling region.

50. A collection of nucleic acid sequences encoding the one or more proteins of the cell according to any of claim 29 to claim 49.

51. The collection of nucleic acid sequences according to claim 50, provided in the form of one or more expression vectors comprising the nucleic acid sequences.

52. A method of treating a condition in a subject in need thereof, the method comprising providing the subject with a cell according to any of claims 29 to 49.

53. The method according to claim 52, wherein the subject is provided directly with a cell according to any of claims 29 to 49.

54. The method according to claim 52, wherein the subject is provided indirectly with a cell according to any of claims 29 to 49.

55. The method according to claim 54, wherein the subject is provided with a collection of nucleic acids according to claim 50 or 51.

56. The method according to any of claims 52 to 55 in the treatment of cancer.

57. The method according to any of claims 52 to 55 in the treatment of viral infection.
